(19) United States
(12) Patent Application Publication
(10) Pub. No.: US 2011/0190309 A1
(43) Pub. Date: Aug. 4, 2011

(54) PHARMACEUTICAL PRODUCT COMPRISING A MUSCARINIC RECEPTOR ANTAGONIST AND A Beta2-ADRENOCEPTOR AGONIST

(76) Inventors: Rhonan Ford, Leicestershire (GB); Andrew Mather, Leicestershire (GB); Antonio Mete, Leicestershire (GB); Katherine Wiley, Leicestershire (GB); Richard James Bull, Essex (GB)

(21) Appl. No.: 12/992,226

(22) PCT Filed: May 12, 2009
(86) PCT No.: PCT/SE2009/050525

§ 371 Date: Apr. 18, 2011

(30) Foreign Application Priority Data
May 13, 2008 (GB) 0808710.8
Jan. 14, 2009 (GB) 0900563.8

(51) Int. Cl. A61K 31/501; A61K 31/497; A61K 31/439; A61P 11/00

(52) U.S. Cl. 514/252.04; 514/255.05; 514/305

(57) ABSTRACT

The invention provides a pharmaceutical product, kit or composition comprising a first active ingredient which is a selected muscarinic receptor antagonist, and a second active ingredient which is a β₂-adrenoceptor agonist, or use in the treatment of respiratory diseases such as chronic obstructive pulmonary disease and asthma.

The present invention relates to combinations of pharmaceutically active substances for use in the treatment of respiratory diseases, especially chronic obstructive pulmonary disease (COPD) and asthma.

The essential function of the lungs requires a fragile structure with enormous exposure to the environment, including pollutants, microbes, allergens, and carcinogens. Host factors, resulting from interactions of lifestyle choices and genetic composition, influence the response to this exposure. Damage or infection to the lungs can give rise to a wide range of diseases of the respiratory system (or respiratory diseases). A number of these diseases are of great public health importance. Respiratory diseases include Acute Lung Injury, Acute Respiratory Distress Syndrome (ARDS), occupational lung disease, lung cancer, tuberculosis, fibrosis, pneumoconiosis, pneumonia, emphysema, Chronic Obstructive Pulmonary Disease (COPD) and asthma.

Among the most common of the respiratory diseases is asthma. Asthma is generally defined as an inflammatory disorder of the airways with clinical symptoms arising from intermittent airflow obstruction. It is characterised clinically by paroxysms of wheezing, dyspnea and cough. It is a chronic disabling disorder that appears to be increasing in prevalence and severity. It is estimated that 15% of children and 5% of adults in the population of developed countries suffer from asthma. Therapy should therefore be aimed at controlling symptoms so that normal life is possible and at the same time provide basis for treating the underlying inflammation.

COPD is a term which refers to a large group of lung diseases which can interfere with normal breathing. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is tobacco smoke. COPD patients have a variety of symptoms, including cough, shortness of breath, and excessive production of sputum; such symptoms arise from dysfunction of a number of cellular compartments, including neutrophils, macrophages, and epithelial cells. The two most important conditions covered by COPD are chronic bronchitis and emphysema.

Chronic bronchitis is a long-standing inflammation of the bronchi which causes increased production of mucous and other changes. The patients' symptoms are cough and expectoration of sputum. Chronic bronchitis can lead to more frequent and severe respiratory infections, narrowing and plugging of the bronchi, difficult breathing and disability.

Emphysema is a chronic lung disease which affects the alveoli and/or the ends of the smallest bronchi. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath.

Therapeutic agents used in the treatment of respiratory diseases include β₂-adrenoceptor agonists. These agents (also known as beta2 (β₂)-agonists) may be used to alleviate symptoms of respiratory diseases by relaxing the bronchial smooth muscles, reducing airway obstruction, reducing lung hyperinflation and decreasing shortness of breath. Compounds currently under evaluation as once-daily β2 agonists are described in Expert Opin. Investig. Drugs 14 (7), 775-783 (2005).

A further class of therapeutic agent used in the treatment of respiratory diseases are muscarinic antagonists. Muscarinic receptors are a G-protein coupled receptor (GPCR) family having five family members M₁, M₂, M₃, M₄ and M₅. Of the five muscarinic subtypes, three (M₁, M₂ and M₃) are known to exert physiological effects on human lung tissue. Parasympathetic nerves are the main pathway for reflex bronchoconstriction in human airways and mediate airway tone by releasing acetylcholine onto muscarinic receptors. Airway tone is increased in patients with respiratory disorders such as asthma and chronic obstructive pulmonary disease (COPD), and for this reason muscarinic receptor antagonists have been developed for use in treating airway diseases. Muscarinic receptor antagonsists, often called anticholinergics in clinical practice, have gained widespread acceptance as a first-line therapy for individuals with COPD, and their use has been extensively reviewed in the literature (e.g. Lee et al, Current Opinion in Pharmacology 2001, 1,223-229).

Whilst treatment with a β₂-adrenoceptor agonist or a muscarinic antagonist can yield important benefits, the efficacy of these agents is often far from satisfactory. Moreover, in view of the complexity of respiratory diseases such as asthma and COPD, it is unlikely that any one mediator can satisfactorily treat the disease alone. Hence there is a pressing medical need for new therapies against respiratory diseases such as COPD and asthma, in particular for therapies with disease modifying potential.

The present invention provides a pharmaceutical product comprising, in combination, a first active ingredient which is a muscarinic antagonist selected from:

(R)-3-(1-Phenyl-cycloheptanecarbonyloxy)-1-(pyrazin-2-ylcarbamoylmethyl)-1-azonia-bicyclo[2.2.2]octane X;
(R)-3-(1-Phenyl-cycloheptanecarbonyloxy)-1-(pyridazin-3-ylcarbamoylmethyl)-1-azonia-bicyclo[2.2.2]octane X;
(R)-3-[1-(3-Fluoro-phenyl)-cycloheptanecarbonyloxy]-1-(pyrazin-2-ylcarbamoylmethyl)-1-azonia-bicyclo[2.2.2]octane X;
(R)-3-[1-(3-Fluoro-phenyl)-cycloheptanecarbonyloxy]-1-(isoxazol-3-ylcarbamoylmethyl)-1-azonia-bicyclo[2.2.2]octane X;
(R)-3-(1-Phenyl-cycloheptanecarbonyloxy)-1-(pyridin-2-ylcarbamoylmethyl)-1-azonia-bicyclo[2.2.2]octane X;
(R)-1-[(5-Fluoro-pyridin-2-ylcarbamoyl)-methyl]-3-(1-phenyl-cycloheptane carb onyloxy)-1-azonia-bicyclo[2.2.2]octane X;
(R)-3-(1-Phenyl-cycloheptane carb onyloxy)-1-(pyridin-3-ylcarbamoylmethyl)-1-azonia-bicyclo[2.2.2]octane X; and
(R)-1-[(2-Methyl-pyridin-4-ylcarbamoyl)-methyl]-3-(1-phenyl-cycloheptanecarbonyloxy)-1-azonia-bicyclo[2.2.2]octane X;
   wherein X represents a pharmaceutically acceptable anion of a mono or polyvalent acid, and a second active ingredient which is a β₂-adrenoceptor agonist.

A beneficial therapeutic effect may be observed in the treatment of respiratory diseases if a muscarinic antagonist according to the present invention is used in combination with a β₂-adrenoceptor agonist. The beneficial effect may be observed when the two active substances are administered simultaneously (either in a single pharmaceutical preparation or via separate preparations), or sequentially or separately via separate pharmaceutical preparations.

The pharmaceutical product of the present invention may, for example, be a pharmaceutical composition comprising the first and second active ingredients in admixture. Alternatively, the pharmaceutical product may, for example, be a kit comprising a preparation of the first active ingredient and a preparation of the second active ingredient and, optionally, instructions for the simultaneous, sequential or separate administration of the preparations to a patient in need thereof.

The first active ingredient in the combination of the present invention is a muscarinic antagonist selected from:
(R)-3-(1-Phenyl-cycloheptanecarbonyloxy)-1-(pyrazin-2-ylcarbamoylmethyl)-1-azonia-bicyclo[2.2.2]octane X;
(R)-3-(1-Phenyl-cycloheptanecarbonyloxy)-1-(pyridazin-3-ylcarbamoylmethyl)-1-azonia-bicyclo[2.2.2]octane X;
(R)-3-[1-(3-Fluoro-phenyl)-cycloheptanecarbonyloxy]-1-(pyrazin-2-ylcarbamoylmethyl)-1-azonia-bicyclo[2.2.2]octane X;
(R)-3-[1-(3-Fluoro-phenyl)-cycloheptanecarbonyloxy]-1-(isoxazol-3-ylcarbamoylmethyl)-1-azonia-bicyclo[2.2.2]octane X;
(R)-3-(1-Phenyl-cycloheptanecarbonyloxy)-1-(pyridin-2-ylcarbamoylmethyl)-1-azonia-bicyclo[2.2.2]octane X;
(R)-1-[(5-Fluoro-pyridin-2-ylcarbamoyl)-methyl]-3-(1-phenyl-cycloheptanecarbonyloxy)-1-azonia-bicyclo[2.2.2]octane X;
(R)-3-(1-Phenyl-cycloheptanecarbonyloxy)-1-(pyridin-3-ylcarbamoylmethyl)-1-azonia-bicyclo[2.2.2]octane X; and
(R)-1-[(2-Methyl-pyridin-4-ylcarbamoyl)-methyl]-3-(1-phenyl-cycloheptanecarbonyloxy)-1-azonia-bicyclo[2.2.2]octane X,
   wherein X represents a pharmaceutically acceptable anion of a mono or polyvalent acid.

The muscarinic antagonists of the invention are selected members of a novel class of compound described in co-pending application PCT/GB2007/004350 (WO2008/059245) which display high potency to the M3 receptor. The names of the muscarinic antagonists are IUPAC names generated by the Beilstein Autonom 2000 naming package, as supplied by MDL Information Systems Inc., based on the structures depicted in the examples, and stereochemistry assigned according to the Cahn-Ingold-Prelog system For example, the name (R)-3-(1-Phenyl-cycloheptanecarbonyloxy)-1-(pyrazin-2-ylcarbamoylmethyl)-1-azonia-bicyclo[2.2.2]octane, was generated from the structure:

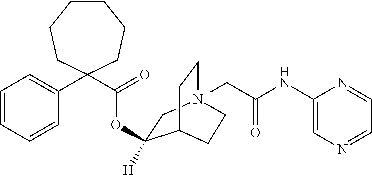

The muscarinic antagonists of the present invention comprise an anion X associated with the positive charge on the quaternary nitrogen atom. The anion X may be any pharmaceutically acceptable anion of a mono or polyvalent (e.g. bivalent) acid. In an embodiment of the invention X may be an anion of a mineral acid, for example chloride, bromide, iodide, sulfate, toluenesulfonate (tosylate), edisylate (ethane-1,2-disulfonate), isethionate (2-hydroxyethylsulfonate), nitrate or phosphate; or an anion of a suitable organic acid, for example acetate, maleate, fumarate, citrate, lactate, oxalate, oleic, succinate, tartrate, methanesulphonate (mesylate), p-toluenesulphonate, benzenesulphonate, napadisylate (naphthalene-1,5-disulphonate) (e.g. a heminapadisylate), maleate ((Z)-3-carboxy-acrylate), succinate (3-carboxy-propionate), malate ((S)-3-carboxy-2-hydroxy-propionate), p-acetamidobenzoate, 2,5-dichlorobenzenesulphonate, 1-hydroxy-2-naphthoate (xinafoate) or 1-hydroxynaphthalene-2-sulphonate.

In an embodiment of the invention, the muscarinic receptor antagonist is selected from:
(R)-3-(1-Phenyl-cycloheptanecarbonyloxy)-1-(pyrazin-2-ylcarbamoylmethyl)-1-azonia-bicyclo[2.2.2]octane bromide;
(R)-3-(1-Phenyl-cycloheptanecarbonyloxy)-1-(pyrazin-2-ylcarbamoylmethyl)-1-azonia-bicyclo[2.2.2]octane chloride;
(R)-3-(1-Phenyl-cycloheptanecarbonyloxy)-1-(pyrazin-2-ylcarbamoylmethyl)-1-azonia-bicyclo[2.2.2]octane 1-hydroxy-naphthalene-2-sulfonate;
(R)-3-(1-Phenyl-cycloheptanecarbonyloxy)-1-(pyrazin-2-ylcarbamoylmethyl)-1-azonia-bicyclo[2.2.2]octane 2,5-dichlorobenzenesulfonate;
(R)-3-(1-Phenyl-cycloheptanecarbonyloxy)-1-(pyrazin-2-ylcarbamoylmethyl)-1-azonia-bicyclo[2.2.2]octane hemi-naphthalene-1,5-disulfonate;
(R)-3-(1-Phenyl-cycloheptane carb onyloxy)-1-(pyridazin-3-ylcarbamoylmethyl)-1-azonia-bicyclo[2.2.2]octane bromide
(R)-3-[1-(3-Fluoro-phenyl)-cycloheptanecarbonyloxy]-1-(pyrazin-2-ylcarbamoylmethyl)-1-azonia-bicyclo[2.2.2]octane bromide;
(R)-3-[1-(3-Fluoro-phenyl)-cycloheptanecarbonyloxy]-1-(is oxazol-3-ylcarbamoylmethyl)-1-azonia-bicyclo[2.2.2]octane bromide;
(R)-3-(1-Phenyl-cycloheptane carb onyloxy)-1-(pyridin-2-ylcarbamoylmethyl)-1-azonia-bicyclo[2.2.2]octane bromide;
(R)-3-(1-Phenyl-cycloheptane carb onyloxy)-1-(pyridin-2-ylcarbamoylmethyl)-1-azonia-bicyclo[2.2.2]octane chloride;
(R)-3-(1-Phenyl-cycloheptane carb onyloxy)-1-(pyridin-2-ylcarbamoylmethyl)-1-azonia-bicyclo[2.2.2]octane hemi-naphthalene-1,5-disulfonate;
(R)-1-[(5-Fluoro-pyridin-2-ylcarbamoyl)-methyl]-3-(1-phenyl-cycloheptane carb onyloxy)-1-azonia-bicyclo[2.2.2]octane chloride;
(R)-3-(1-Phenyl-cycloheptane carb onyloxy)-1-(pyridin-3-ylcarbamoylmethyl)-1-azonia-bicyclo[2.2.2]octane chloride; and
(R)-1-[(2-Methyl-pyridin-4-ylcarbamoyl)-methyl]-3-(1-phenyl-cycloheptanecarbonyloxy)-1-azonia-bicyclo[2.2.2]octane chloride.

In an embodiment of the invention, the muscarinic receptor antagonist is in the form of a bromide or napadisylate salt.

In an embodiment of the invention, the muscarinic receptor antagonist is in the form of a napadisylate salt. When the muscarinic antagonist is a napadisylate salt the cation/anion ratio may vary, and for example may be 1:1 or 2:1 or a value between 1:1 and 2:1.

In an embodiment of the invention the muscarinic antagonist is in the form of a napadisylate salt wherein the napadisylate salt cation/anion ratio is 2:1.i.e. a hemi-napadisylate. Examples of muscarinic antagonists according to this embodiment include:
(R)-3-(1-Phenyl-cycloheptanecarbonyloxy)-1-(pyrazin-2-ylcarbamoylmethyl)-1-azonia-bicyclo[2.2.2]octane hemi-naphthalene-1,5-disulfonate; and
(R)-3-(1-Phenyl-cycloheptanecarbonyloxy)-1-(pyridin-2-ylcarbamoylmethyl)-1-azonia-bicyclo[2.2.2]octane hemi-naphthalene-1,5-disulfonate.

In an embodiment of the invention, the muscarinic receptor antagonist is in the form of a 2,5-dichlorobenzene sulphonate or 1-hydroxynaphthalene-2-sulphonate salt.

Examples of muscarinic antagonists according to this embodiment include
(R)-3-(1-Phenyl-cycloheptanecarbonyloxy)-1-(pyrazin-2-ylcarbamoylmethyl)-1-azonia-bicyclo[2.2.2]octane 1-hydroxy-naphthalene-2-sulfonate; and
(R)-3-(1-Phenyl-cycloheptanecarbonyloxy)-1-(pyrazin-2-ylcarbamoylmethyl)-1-azonia-bicyclo[2.2.2]octane 2,5-dichlorobenzenesulfonate.

In an embodiment of the invention, the muscarinic receptor antagonist is in the form of a bromide salt.

The second active ingredient in the combination of the present invention is a β₂-adrenoceptor agonist. The β₂-adrenoceptor agonist of the present invention may be any compound or substance capable of stimulating the β₂-receptors and acting as a is bronchodilator. In the context of the present specification, unless otherwise stated, any reference to a β₂-adrenoceptor agonist includes active salts, solvates or derivatives that may be formed from said β₂-adrenoceptor agonist and any enantiomers and mixtures thereof. Examples of possible salts or derivatives of β₂-adrenoceptor agonist are acid addition salts such as the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid, maleic acid, trifluoroacetic acid, D-mandelate and pharmaceutically acceptable esters (e.g. C₁-C₆ alkyl esters). The β₂-agonists may also be in the form of solvates, e.g. hydrates.

Examples of a β₂-adrenoceptor agonist that may be used in the pharmaceutical product according to this embodiment include metaproterenol, isoproterenol, isoprenaline, albuterol, salbutamol (e.g. as sulphate), formoterol (e.g. as fumarate), salmeterol (e.g. as xinafoate), terbutaline, orciprenaline, bitolterol (e.g. as mesylate), pirbuterol or indacaterol. The β₂-adrenoceptor agonist of this embodiment may be a long-acting β₂-agonist (i.e. a β₂-agonist with activity that persists for more than 24 hours), for example salmeterol (e.g. as xinafoate), formoterol (e.g. as fumarate), bambuterol (e.g. as hydrochloride), carmoterol (TA 2005, chemically identified as 2(1H)-Quinolone, 8-hydroxy-5-[1-hydroxy-2-[[2-(4-methoxy-phenyl)-1-methylethyl]-amino]ethyl]-monohydrochloride, [R—(R*,R*)] also identified by Chemical Abstract Service Registry Number 137888-11-0 and disclosed in U.S. Pat. No. 4,579,854), indacaterol (CAS no 312753-06-3; QAB-149), formanilide derivatives e.g. 3-(4-{[6-({(2R)-2-[3-(formylamino)-4-hydroxyphenyl]-2-hydroxyethyl}amino)hexyl]oxy}-butyl)-benzenesulfonamide as disclosed in WO 2002/76933, benzenesulfonamide derivatives e.g. 3-(4-{[6-({(2R)-2-hydroxy-2-[4-hydroxy-3-(hydroxy-methyl)phenyl]ethyl}amino)-hexyl]oxy}butyl)benzenesulfonamide as disclosed in WO 2002/88167, aryl aniline receptor agonists as disclosed in WO 2003/042164 and WO 2005/025555, indole derivatives as disclosed in WO 2004/032921, in US 2005/222144, compounds GSK 159797, GSK 159802, GSK 597901, GSK 642444 and GSK 678007.

In an embodiment of the present invention, the β₂-adrenoceptor agonist is formoterol. The chemical name for formoterol is N-[2-hydroxy-5-[(1)-1-hydroxy-2-[[(1)-2-(4-methoxyphenyl)-1-methylethyl]amino]ethyl]phenyl]-formamide. The preparation of formoterol is described, for example, in WO 92/05147. In one aspect of this embodiment, the β₂-adrenoceptor agonist is formoterol fumarate. It will be understood that the invention encompasses the use of all optical isomers of formoterol and mixtures thereof including racemates. Thus for example, the term formoterol encompasses N-[2-hydroxy-5-[(1R)-1-hydroxy-2-[[(1R)-2-(4-methoxyphenyl)-1-methylethyl]amino]ethyl]phenyl]-formamide, N-[2-hydroxy-5-[(1S)-1-hydroxy-2-[[(1S)-2-(4-methoxyphenyl)-1-methylethyl]amino]ethyl]phenyl]-formamide and a mixture of such enantiomers, including a racemate.

In an embodiment of the invention, the β₂-adrenoceptor agonist is selected from:
N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide,
N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide, and
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one,
   or a pharmaceutically acceptable salt thereof. The β₂-adrenoceptor agonists according to this embodiment may be prepared as described in the experimental preparation section of the present application. The names of the β₂-adrenoceptor agonists of this embodiment are IUPAC names generated by the IUPAC NAME, ACD Labs Version 8 naming package.

In a further embodiment of the invention, the β₂-adrenoceptor agonist is selected from:
N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide,
N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide dihydrobromide, and
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one dihydrobromide.

In an embodiment of the invention, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane X, and the β₂-adrenoceptor agonist is formoterol (e.g. as fumarate). In one aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane bromide. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane chloride. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane 1-hydroxy-naphthalene-2-sulfonate. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane 2,5-dichlorobenzenesulfonate. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane hemi-naphthalene-1,5-disulfonate.

In an embodiment of the invention, the muscarinic receptor antagonist is (R)-3-[1-(3-Fluoro-phenyl)-cycloheptanecarbonyloxy]-1-(is oxazol-3-ylcarbamoylmethyl)-1-azonia-bicyclo[2.2.2]octane X, and the β₂-adrenoceptor agonist is formoterol (e.g. as fumarate). In one aspect of this embodiment, the muscarinic receptor antagonist is (R)-3-[1-(3-Fluoro-phenyl)-cycloheptanecarbonyloxy]-1-(is oxazol-3-ylcarbamoylmethyl)-1-azonia-bicyclo [2.2.2]octane bromide.

In an embodiment of the invention, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane X, and the β₂-adrenoceptor agonist is formoterol (e.g. as fumarate). In one aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane bromide. In another aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane chloride. In another aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane hemi-naphthalene-1,5-disulfonate.

In an embodiment of the invention, the muscarinic receptor antagonist is (R)-1-[(5-Fluoro-pyridin-2-ylcarbamoyl)-methyl]-3-(1-phenyl-cycloheptanecarbonyloxy)-1-azonia-bicyclo[2.2.2]octane X, and the β₂-adrenoceptor agonist is formoterol (e.g. as fumarate). In one aspect of this embodiment, the muscarinic receptor antagonist is (R)-1-[(5-Fluoro-pyridin-2-ylcarbamoyl)-methyl]-3-(1-phenyl-cycloheptanecarbonyloxy)-1-azonia-bicyclo[2.2.2]octane chloride.

In an embodiment of the invention, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane X, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane bromide. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane chloride In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane 1-hydroxy-naphthalene-2-sulfonate. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane 2,5-dichlorobenzenesulfonate. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane hemi-naphthalene-1,5-disulfonate.

In an embodiment of the invention, the muscarinic receptor antagonist is (R)-3-[1-(3-Fluoro-phenyl)-cycloheptanecarbonyloxy]-1-(isoxazol-3-ylcarbamoylmethyl)-1-azonia-bicyclo[2.2.2]octane X, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is (R)-3-[1-(3-Fluoro-phenyl)-cycloheptanecarbonyloxy]-1-(isoxazol-3-ylcarbamoylmethyl)-1-azonia-bicyclo[2.2.2]octane bromide.

In an embodiment of the invention, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane X, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane bromide. In another aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane chloride. In another aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane hemi-naphthalene-1,5-disulfonate.

In an embodiment the present invention provides a pharmaceutical product, comprising, in combination, a first active ingredient which is (R)-3-(1-Phenyl-cycloheptanecarbonyloxy)-1-(pyridin-2-ylcarbamoylmethyl)-1-azonia-bicyclo[2.2.2]octane X wherein X represents a pharmaceutically acceptable anion of a mono or polyvalent acid, and a second active ingredient which is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof.

In an embodiment of the invention, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane bromide and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide.

In an embodiment of the invention, the muscarinic receptor antagonist is (R)-1-[(5-Fluoro-pyridin-2-ylcarbamoyl)-methyl]-3-(1-phenyl-cycloheptanecarbonyloxy)-1-azonia-bicyclo[2.2.2]octane X, and the β₂-adrenoceptor agonist is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof (e.g. dihydrobromide). In one aspect of this embodiment, the muscarinic receptor antagonist is (R)-1-[(5-Fluoro-pyridin-2-ylcarbamoyl)-methyl]-3-(1-phenyl-cycloheptanecarbonyloxy)-1-azonia-bicyclo[2.2.2]octane chloride.

In an embodiment of the invention, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane X, and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane bromide. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane chloride. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane 1-hydroxy-naphthalene-2-sulfonate. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane 2,5-dichlorobenzenesulfonate. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane hemi-naphthalene-1,5-disulfonate.

In an embodiment of the invention, the muscarinic receptor antagonist is (R)-3-[1-(3-Fluoro-phenyl)-cycloheptanecarbonyloxy]-1-(isoxazol-3-ylcarbamoylmethyl)-1-azonia-bicyclo[2.2.2]octane X, and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is (R)-3-[1-(3-Fluoro-phenyl)-cycloheptanecarbonyloxy]-1-(isoxazol-3-ylcarbamoylmethyl)-1-azonia-bicyclo[2.2.2]octane bromide.

In an embodiment of the invention, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane X, and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane bromide. In another aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane chloride. In another aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane hemi-naphthalene-1,5-disulfonate.

In an embodiment of the invention, the muscarinic receptor antagonist is (R)-1-[(5-Fluoro-pyridin-2-ylcarbamoyl)-methyl]-3-(1-phenyl-cycloheptanecarbonyloxy)-1-azonia-bicyclo[2.2.2]octane X, and the β₂-adrenoceptor agonist is 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one or a pharmaceutically acceptable salt thereof (e.g. dihydrobromide). In one aspect of this embodiment, the muscarinic receptor antagonist is (R)-1-[(5-Fluoro-pyridin-2-ylcarbamoyl)-methyl]-3-(1-phenyl-cycloheptanecarbonyloxy)-1-azonia-bicyclo[2.2.2]octane chloride.

In an embodiment of the invention, the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof. The β₂-adrenoceptor agonist according to this embodiment may be prepared as described in WO2008/075026 A1. In a further aspect of this embodiment, the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide bis-trifluoroacetic acid salt. In a further aspect of this embodiment, the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide dihydrobromide salt. In a further aspect of this embodiment, the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide di-D-mandelate salt.

In an embodiment of the invention, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane X, and the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide or a pharmaceutically acceptable salt thereof (e.g. di-D-mandelate salt). In one aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane bromide. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane chloride. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane 1-hydroxy-naphthalene-2-sulfonate. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane 2,5-dichlorobenzenesulfonate. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane hemi-naphthalene-1,5-disulfonate.

In an embodiment of the invention, the muscarinic receptor antagonist is (R)-3-[1-(3-Fluoro-phenyl)-cycloheptanecarbonyloxy]-1-(isoxazol-3-ylcarbamoylmethyl)-1-azonia-bicyclo[2.2.2]octane X, and the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide or a pharmaceutically acceptable salt thereof (e.g. di-D-mandelate salt). In one aspect of this embodiment, the muscarinic receptor antagonist is (R)-3-[1-(3-Fluoro-phenyl)-cycloheptanecarbonyloxy]-1-(isoxazol-3-ylcarbamoylmethyl)-1-azonia-bicyclo[2.2.2]octane bromide.

In an embodiment of the invention, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane X, and the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide or a pharmaceutically acceptable salt thereof (e.g. di-D-mandelate salt). In one aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane bromide. In another aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane chloride. In another aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane hemi-naphthalene-1,5-disulfonate.

In an embodiment the present invention provides a pharmaceutical product, comprising, in combination, a first active ingredient which is (R)-3-(1-Phenyl-cycloheptanecarbonyloxy)-1-(pyridin-2-ylcarbamoylmethyl)-1-azonia-bicyclo[2.2.2]octane X wherein X represents a pharmaceutically acceptable anion of a mono or polyvalent acid, and a second active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof.

In an embodiment of the invention, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane bromide, and the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide di-D-mandelate salt.

In an embodiment of the invention, the muscarinic receptor antagonist is (R)-1-[(5-Fluoro-pyridin-2-ylcarbamoyl)-methyl]-3-(1-phenyl-cycloheptanecarbonyloxy)-1-azonia-bicyclo[2.2.2]octane X, and the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide or a pharmaceutically acceptable salt thereof (e.g. di-D-mandelate salt). In one aspect of this embodiment, the muscarinic receptor antagonist is (R)-1-[(5-Fluoro-pyridin-2-ylcarbamoyl)-methyl]-3-(1-phenyl-cycloheptane carb onyloxy)-1-azonia-bicyclo[2.2.2]octane chloride.

In an embodiment of the invention, the β₂-adrenoceptor agonist is indacaterol.

In an embodiment of the invention, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane X, and the β₂-adrenoceptor agonist is indacaterol. In one aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane bromide. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane chloride. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane 1-hydroxy-naphthalene-2-sulfonate. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane 2,5-dichlorobenzenesulfonate. In another aspect of this embodiment, the muscarinic receptor antagonist is (R)-3-(1-Phenyl-cycloheptanecarbonyloxy)-1-(pyrazin-2-ylcarbamoylmethyl)-1-azonia-bicyclo[2.2.2]octane hemi-naphthalene-1,5-disulfonate.

In an embodiment of the invention, the muscarinic receptor antagonist is (R)-3-[1-(3-Fluoro-phenyl)-cycloheptanecarbonyloxy]-1-(isoxazol-3-ylcarbamoylmethyl)-1-azonia-bicyclo[2.2.2]octane X, and the β₂-adrenoceptor agonist is indacaterol. In one aspect of this embodiment, the muscarinic receptor antagonist is (R)-3-[1-(3-Fluoro-phenyl)-cycloheptanecarbonyloxy]-1-(isoxazol-3-ylcarbamoylmethyl)-1-azonia-bicyclo[2.2.2]octane bromide.

In an embodiment of the invention, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane X, and the β₂-adrenoceptor agonist is indacaterol. In one aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane bromide. In another aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane chloride. In another aspect of this embodiment, the muscarinic receptor antagonist is (R)-3-(1-Phenyl-cycloheptanecarbonyloxy)-1-(pyridin-2-ylcarbamoylmethyl)-1-azonia-bicyclo[2.2.2]octane hemi-naphthalene-1,5-disulfonate.

In an embodiment of the invention, the muscarinic receptor antagonist is (R)-1-[(5-Fluoro-pyridin-2-ylcarbamoyl)-methyl]-3-(1-phenyl-cycloheptanecarbonyloxy)-1-azonia-bicyclo[2.2.2]octane X, and the β₂-adrenoceptor agonist is indacaterol. In one aspect of this embodiment, the muscarinic receptor antagonist is (R)-1-[(5-Fluoro-pyridin-2-ylcarbamoyl)-methyl]-3-(1-phenyl-cycloheptanecarbonyloxy)-1-azonia-bicyclo[2.2.2]octane chloride.

The combination of the present invention may provide a beneficial therapeutic effect in the treatment of respiratory diseases. Examples of such possible effects include improvements in one or more of the following parameters: reducing inflammatory cell influx into the lung, mild and severe exacerbations, FEV₁ (forced expiratory volume in one second), vital capacity (VC), peak expiratory flow (PEF), symptom scores and Quality of Life.

The muscarinic antagonist (first active ingredient) and β₂-adrenoceptor agonist (second active ingredient) of the present invention may be administered simultaneously, sequentially or separately to treat respiratory diseases. By sequential it is meant that the active ingredients are administered, in any order, one immediately after the other. They may still have the desired effect if they are administered separately, but when administered in this manner they will generally be administered less than 4 hours apart, more conveniently less than two hours apart, more conveniently less than 30 minutes apart and most conveniently less than 10 minutes apart.

The active ingredients of the present invention may be administered by oral or parenteral (e.g. intravenous, subcutaneous, intramuscular or intraarticular) administration using conventional systemic dosage forms, such as tablets, capsules, pills, powders, aqueous or oily solutions or suspensions, emulsions and sterile injectable aqueous or oily solutions or suspensions. The active ingredients may also be administered topically (to the lung and/or airways) in the form of solutions, suspensions, aerosols and dry powder formulations. These dosage forms will usually include one or more pharmaceutically acceptable ingredients which may be selected, for example, from adjuvants, carriers, binders, lubricants, diluents, stabilising agents, buffering agents, emulsifying agents, viscosity-regulating agents, surfactants, preservatives, flavourings and colorants. As will be understood by those skilled in the art, the most appropriate method of administering the active ingredients is dependent on a number of factors.

In one embodiment of the present invention the active ingredients are administered via separate pharmaceutical preparations. Therefore, in one aspect, the present invention provides a kit comprising a preparation of a first active ingredient which is a muscarinic antagonist according to the present invention, and a preparation of a second active ingredient which is a β₂-adrenoceptor agonist, and optionally instructions for the simultaneous, sequential or separate administration of the preparations to a patient in need thereof.

In another embodiment the active ingredients may be administered via a single pharmaceutical composition. Therefore, the present invention further provides a pharmaceutical composition comprising, in admixture, a first active ingredient, which is a muscarinic antagonist according to the present invention, and a second active ingredient, which is a β₂-adrenoceptor agonist.

The pharmaceutical compositions of the present invention may be prepared by mixing the muscarinic antagonist (first active ingredient) with a β₂-adrenoceptor agonist (second active ingredient) and a pharmaceutically acceptable adjuvant, diluent or carrier. Therefore, in a further aspect of the present invention there is provided a process for the preparation of a pharmaceutical composition, which comprises mixing a muscarinic antagonist according to the present invention with a β₂-adrenoceptor agonist and a pharmaceutically acceptable adjuvant, diluent or carrier.

It will be understood that the therapeutic dose of each active ingredient administered in accordance with the present invention will vary depending upon the particular active ingredient employed, the mode by which the active ingredient is to be administered, and the condition or disorder to be treated.

In one embodiment of the present invention, muscarinic antagonist according to the present invention is administered via inhalation. When administered via inhalation the dose of the muscarinic antagonist according to the present invention will generally be in the range of from 0.1 microgram (μg) to 5000 μg, 0.1 to 1000 μg, 0.1 to 500 μg, 0.1 to 100 μg, 0.1 to 50 μg, 0.1 to 5 μg, 5 to 5000 μg, 5 to 1000 μg, 5 to 500 μg, 5 to 100 μg, 5 to 50 μg, 5 to 10 μg, 10 to 5000 μg, 10 to 1000 μg, 10 to 500 μg, 10 to 100 μg, 10 to 50 μg, 20 to 5000 μg, 20 to 1000 μg, 20 to 500 μg, 20 to 100 μg, 20 to 50 μg, 50 to 5000 μg, 50 to 1000 μg, 50 to 500 μg, 50 to 100 μg, 100 to 5000 μg, 100 to 1000 μg or 100 to 500 μg. The dose will generally be administered from 1 to 4 times a day, conveniently once or twice a day, and most conveniently once a day.

In one embodiment of the present invention the β₂-adrenoceptor agonist may conveniently be administered by inhalation. When administered via inhalation the dose of the β₂-agonist will generally be in the range of from 0.1 to 50 μg, 0.1 to 40 μg, 0.1 to 30 μg, 0.1 to 20 μg, 0.1 to 10 μg, 5 to 10 μg, 5 to 50 μg, 5 to 40 μg, 5 to 30 μg, 5 to 20 μg, 5 to 10 μg, 10 to 50 μg, 10 to 40 μg 10 to 30 μg, or 10 to 20 μg. The dose will generally be administered from 1 to 4 times a day, conveniently once or twice a day, and most conveniently once a day.

In one embodiment, the present invention provides a pharmaceutical product comprising, in combination, a first active ingredient which is a muscarinic antagonist according to the present invention, and a second active ingredient which is a β₂-adrenoceptor agonist, wherein each active ingredient is formulated for inhaled administration.

The active ingredients of the present invention are conveniently administered via inhalation (e.g. topically to the lung and/or airways) in the form of solutions, suspensions, aerosols and dry powder formulations. For example metered dose inhaler devices may be used to administer the active ingredients, dispersed in a suitable propellant and with or without additional excipients such as ethanol, surfactants, lubricants or stabilising agents. Suitable propellants include hydrocarbon, chlorofluorocarbon and hydrofluoroalkane (e.g. heptafluoroalkane) propellants, or mixtures of any such propellants. Preferred propellants are P134a and P227, each of which may be used alone or in combination with other propellants and/or surfactant and/or other excipients. Nebulised aqueous suspensions or, preferably, solutions may also be employed, with or without a suitable pH and/or tonicity adjustment, either as a unit-dose or multi-dose formulations.

Dry powder formulations and pressurized HFA aerosols of the active ingredients may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

The combination of the present invention is useful in the treatment or prevention of respiratory-tract disorders such as chronic obstructive pulmonary disease (COPD), chronic bronchitis of all types (including dyspnoea associated therewith), asthma (allergic and non-allergic; ‘wheezy-infant syndrome’), adult/acute respiratory distress syndrome (ARDS), chronic respiratory obstruction, bronchial hyperactivity, pulmonary fibrosis, pulmonary emphysema, and allergic rhinitis, exacerbation of airway hyperreactivity consequent to other drug therapy, particularly other inhaled drug therapy or pneumoconiosis (for example aluminosis, anthracosis, asbestosis, chalicosis, ptilosis, siderosis, silicosis, tabacosis and byssinosis).

Dry powder inhalers may be used to administer the active ingredients, alone or in combination with a pharmaceutically acceptable carrier, in the later case either as a finely divided powder or as an ordered mixture. The dry powder inhaler may be single dose or multi-dose and may utilise a dry powder or a powder-containing capsule.

Metered dose inhaler, nebuliser and dry powder inhaler devices are well known and a variety of such devices are available.

The present invention further provides a pharmaceutical product, kit or pharmaceutical composition according to the invention for simultaneous, sequential or separate use in therapy.

The present invention further provides the use of a pharmaceutical product, kit or pharmaceutical composition according to the invention in the treatment of a respiratory disease, in particular chronic obstructive pulmonary disease or asthma.

The present invention further provides the use of a pharmaceutical product, kit or pharmaceutical composition according to the invention in the manufacture of a medicament for the treatment of a respiratory disease, in particular chronic obstructive pulmonary disease or asthma.

The present invention still further provides a method of treating a respiratory disease which comprises simultaneously, sequentially or separately administering:

(a) a (therapeutically effective) dose of a first active ingredient which is a muscarinic antagonist according to the present invention; and
(b) a (therapeutically effective) dose of a second active ingredient which is a β₂-adrenoceptor agonist; to a patient in need thereof.

In the context of the present specification, the term “therapy” also includes “prophylaxis” unless there are specific indications to the contrary. The terms “therapeutic” and “therapeutically” should be construed accordingly. Prophylaxis is expected to be particularly relevant to the treatment of persons who have suffered a previous episode of, or are otherwise considered to be at increased risk of, the condition or disorder in question. Persons at risk of developing a particular condition or disorder generally include those having a family history of the condition or disorder, or those who have been identified by genetic testing or screening to be particularly susceptible to developing the condition or disorder.

The pharmaceutical product, kit or composition of the present invention may optionally comprise a third active ingredient which third active ingredient is a substance suitable for use in the treatment of respiratory diseases. Examples of a third active ingredient that may be incorporated into the present invention include
a phosphodiesterase inhibitor,
a modulator of chemokine receptor function,
an inhibitor of kinase function,
a protease inhibitor,
a steroidal glucocorticoid receptor agonist, and a
a non-steroidal glucocorticoid receptor agonist.

Examples of a phosphodiesterase inhibitor that may be used as a third active ingredient according to this embodiment include a PDE4 inhibitor such as an inhibitor of the isoform PDE4D, a PDE3 inhibitor and a PDE5 inhibitor. Examples include the compounds
(Z)-3-(3,5-dichloro-4-pyridyl)-2-[4-(2-indanyloxy-5-methoxy-2-pyridyl]propenenitrile,
N-[9-amino-4-oxo-1-phenyl-3,4,6,7-tetrahydropyrrolo[3,2,1-jk][1,4]benzodiazepin-3(R)-yl]pyridine-3-carboxamide (CI-1044),
3-(benzyloxy)-1-(4-fluorobenzyl)-N-[3-(methylsulphonyl)phenyl]-1H-indole-2-carboxamide,
(1S-exo)-5-[3-(bicyclo[2.2.1]hept-2-yloxy)-4-methoxyphenyl]tetrahydro-2(1H)-pyrimidinone (Atizoram),
N-(3,5,dichloro-4-pyridinyl)-2-[1-(4-fluorobenzyl)-5-hydroxy-1H-indol-3-yl]-2-oxoacetamide (AWD-12-281),
β-[3-(cyclopentyloxy)-4-methoxyphenyl]-1,3-dihydro-1,3-dioxo-2H-isoindole-2-propanamide (CDC-801),
N-[9-methyl-4-oxo-1-phenyl-3,4,6,7-tetrahydropyrrolo[3,2,1-jk][1,4]benzodiazepin-3(R)-yl]pyridine-4-carboxamide (CI-1018),
cis-[4-cyano-4-(3-cyclopentyloxy-4-methoxyphenyl)cyclohexane-1-carboxylic acid (Cilomilast),
8-amino-1,3-bis(cyclopropylmethyl)xanthine (Cipamfylline),
N-(2,5-dichloro-3-pyridinyl)-8-methoxy-5-quinolinecarboxamide (D-4418),
5-(3,5-di-tert-butyl-4-hydroxybenzylidene)-2-iminothiazolidin-4-one (Darbufelone),
2-methyl-1-[2-(1-methylethyl)pyrazolo[1,5-a]pyridin-3-yl]-1-propanone (Ibudilast),
2-(2,4-dichlorophenylcarbonyl)-3-ureidobenzofuran-6-ylmethanesulphonate (Lirimilast),
(−)-(R)-5-(4-methoxy-3-propoxyphenyl)-5-methyloxazolidin-2-one (Mesopram),
(−)-cis-9-ethoxy-8-methoxy-2-methyl-1,2,3,4,4a,10b-hexahydro-6-(4-diisopropylaminocarbonylphenyl)-benzo[c][1,6]naphthyridine (Pumafentrine),
3-(cyclopropylmethoxy)-N-(3,5-dichloro-4-pyridyl)-4-(difluoromethoxy)benzamide (Roflumilast),

### the N-oxide of Roflumilast,

5,6-diethoxybenzo[b]thiophene-2-carboxylic acid (Tibenelast),
2,3,6,7-tetrahydro-2-(mesitylimino)-9,10-dimethoxy-3-methyl-4H-pyrimido[6,1-a]isoquinolin-4-one (trequinsin), and

3-[[3-(cyclopentyloxy)-4-methoxyphenyl]-methyl]-N-ethyl-8-(1-methylethyl)-3H-purine-6-amine (V-11294A).

Examples of a modulator of chemokine receptor function that may be used as a third active ingredient according to this embodiment include a CCR3 receptor antagonist, a CCR4 receptor antagonist, a CCR5 receptor antagonist and a CCR8 receptor antagonist.

Examples of an inhibitor of kinase function that may be used as a third active ingredient according to this embodiment include a p38 kinase inhibitor and an IKK inhibitor.

Examples of a protease inhibitor that may be used as a third active ingredient according to this embodiment include an inhibitor of neutrophil elastase or an inhibitor of MMP12.

Examples of a steroidal glucocorticoid receptor agonist that may be used as a third active ingredient according to this embodiment include budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (as e.g. dicloacetate), triamcinolone (e.g. as acetonide), flunisolide, zoticasone, flumoxonide, rofleponide, butixocort (e.g. as propionate ester), prednisolone, prednisone, tipredane, steroid esters e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl)ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, steroid esters according to DE 4129535, steroids according to WO 2002/00679, WO 2005/041980, or steroids GSK 870086, GSK 685698 and GSK 799943.

Examples of a modulator of a non-steroidal glucocorticoid receptor agonist that may be used as a third active ingredient according to this embodiment include those described in WO2006/046916.

The invention is illustrated by the following non-limiting Examples. In the Examples the following Figures are presented:

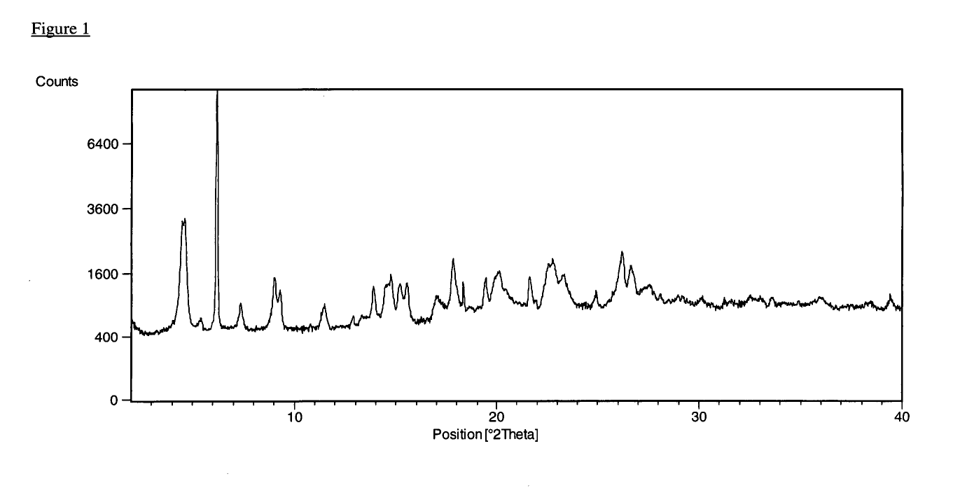

FIG. 1: X-ray powder diffraction pattern of muscarinic antagonist (R)-3-(1-Phenyl-cycloheptanecarbonyloxy)-1-(pyrazin-2-ylcarbamoylmethyl)-1-azonia-bicyclo[2.2.2]octane bromide Crystalline Form A (Example 1).

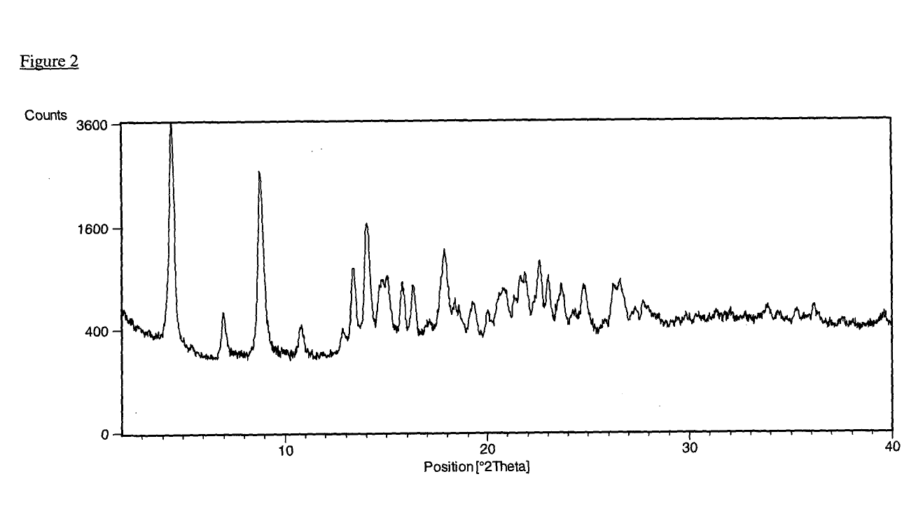

FIG. 2: X-ray powder diffraction pattern of muscarinic antagonist (R)-3-(1-Phenyl-cycloheptanecarbonyloxy)-1-(pyrazin-2-ylcarbamoylmethyl)-1-azonia-bicyclo[2.2.2]octane chloride Crystalline Form A (Example 2).

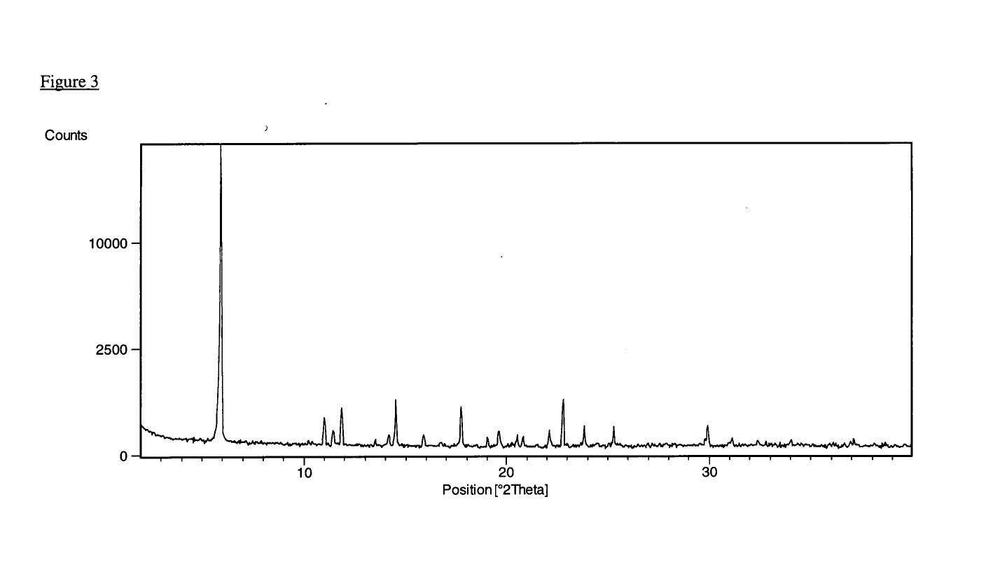

FIG. 3: X-ray powder diffraction pattern of muscarinic antagonist (R)-3-(1-Phenyl-cycloheptanecarbonyloxy)-1-(pyridin-2-ylcarbamoylmethyl)-1-azonia-bicyclo[2.2.2]octane chloride Crystalline Form A (Example 3).

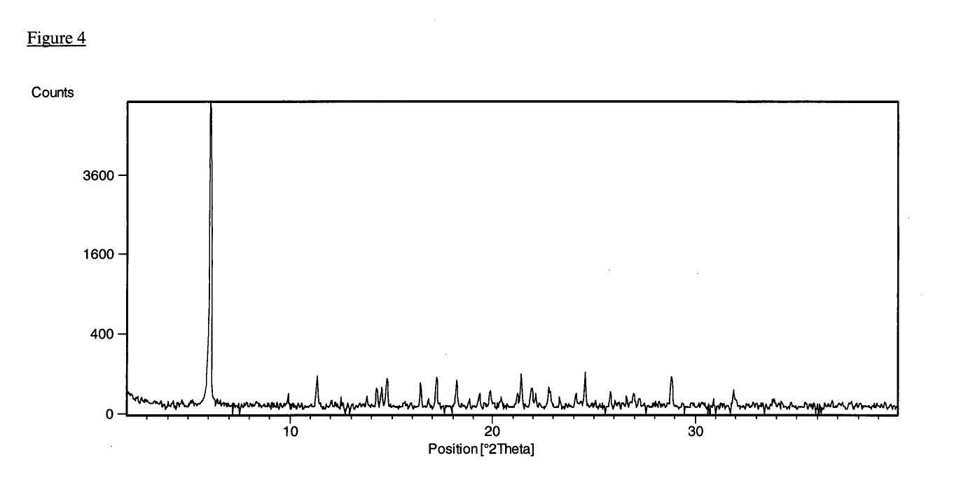

FIG. 4: X-ray powder diffraction pattern of muscarinic antagonist (R)-3-(1-Phenyl-cycloheptanecarbonyloxy)-1-(pyridin-2-ylcarbamoylmethyl)-1-azonia-bicyclo[2.2.2]octane bromide Crystalline Form A (Example 4).

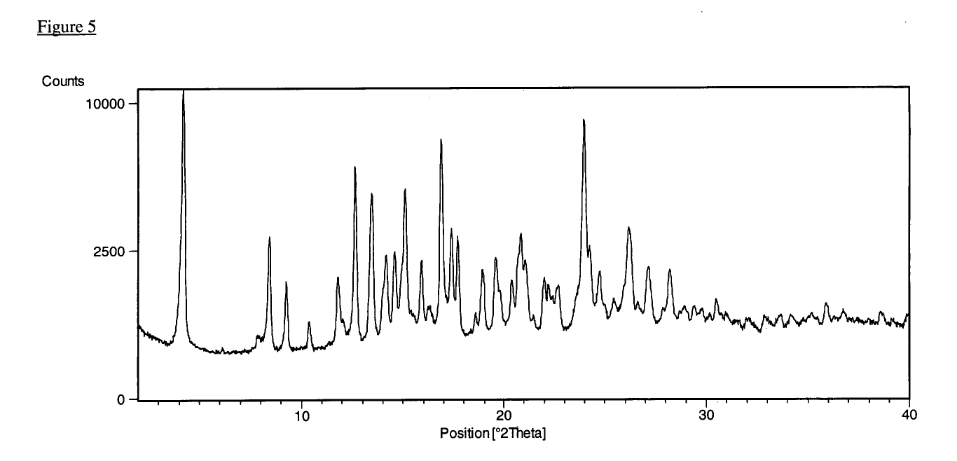

FIG. 5: X-ray powder diffraction pattern of muscarinic antagonist (R)-3-(1-Phenyl-cycloheptanecarbonyloxy)-1-(pyrazin-2-ylcarbamoylmethyl)-1-azonia-bicyclo[2.2.2]octane 1-hydroxy-naphthalene-2-sulfonate Crystalline Form A (Example 5).

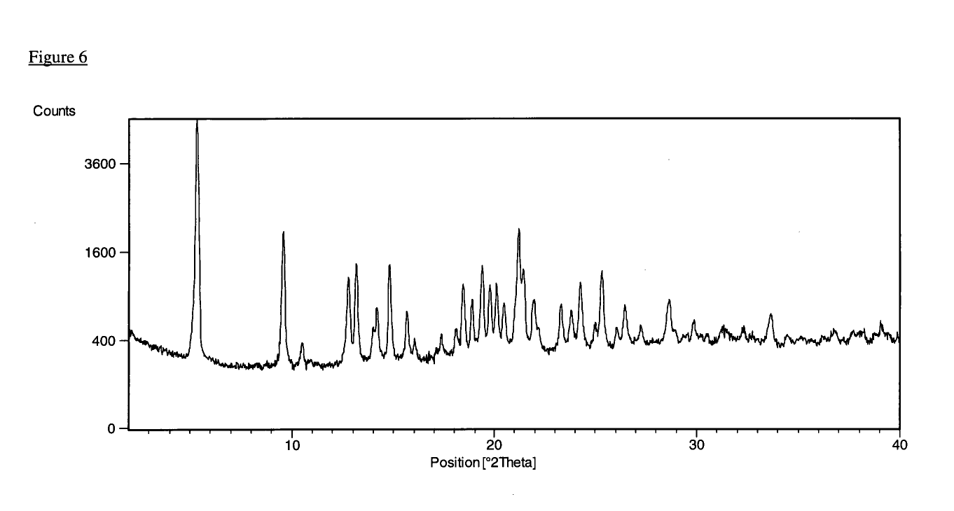

FIG. 6: X-ray powder diffraction pattern of muscarinic antagonist (R)-3-(1-Phenyl-cycloheptanecarbonyloxy)-1-(pyrazin-2-ylcarbamoylmethyl)-1-azonia-bicyclo[2.2.2]octane 2,5-dichloro-benzenesulfonate Crystalline Form A (Example 6).

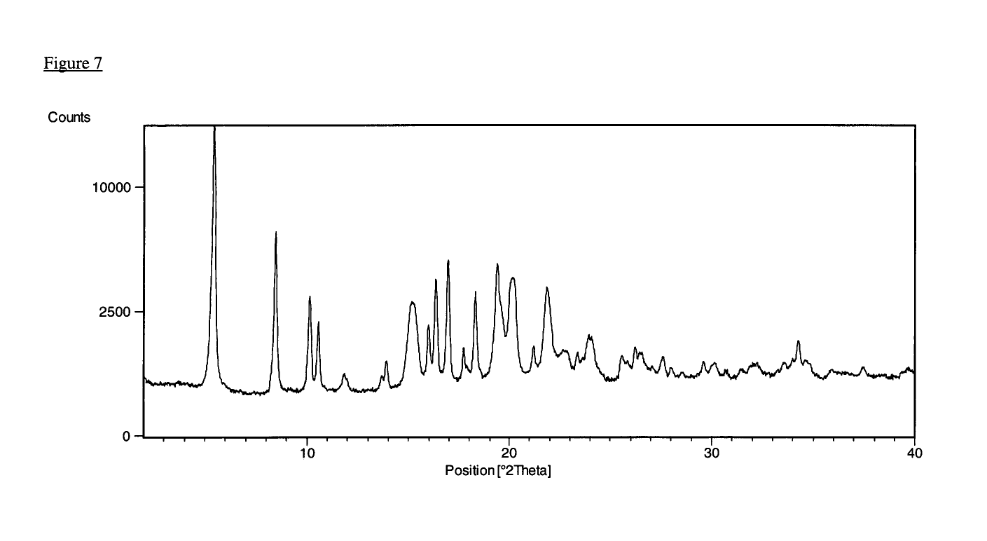

FIG. 7: X-ray powder diffraction pattern of muscarinic antagonist (R)-3-(1-Phenyl-cycloheptanecarbonyloxy)-1-(pyrazin-2-ylcarbamoylmethyl)-1-azonia-bicyclo[2.2.2]octane hemi-naphthalene-1,5-disulfonate Crystalline Form A (Example 7).

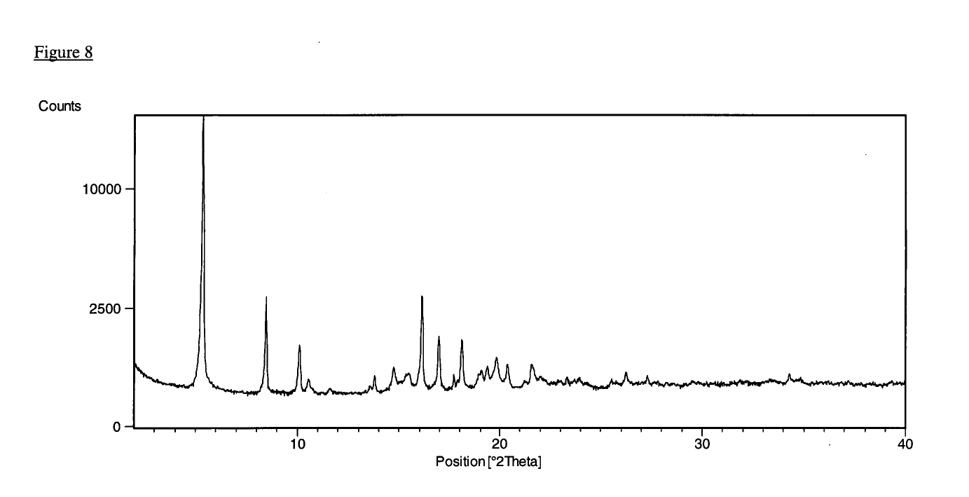

FIG. 8: X-ray powder diffraction pattern of muscarinic antagonist (R)-3-(1-Phenyl-cycloheptanecarbonyloxy)-1-(pyridin-2-ylcarbamoylmethyl)-1-azonia-bicyclo[2.2.2]octane hemi-naphthalene-1,5-disulfonate Crystalline Form A (Example 14).

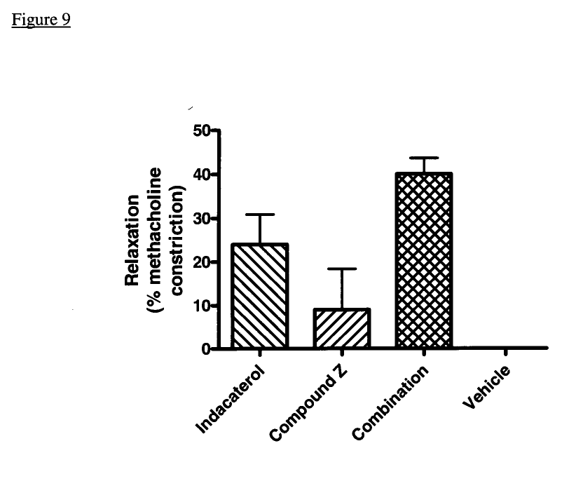

FIG. 9: Percentage relaxation to indacaterol (10 nM), (R)-3-(1-Phenyl-cycloheptanecarbonyloxy)-1-(pyridin-2-ylcarbamoylmethyl)-1-azonia-bicyclo[2.2.2]octane bromide (compound Z) (1 nM) and the combination of indacaterol (10 nM) and (R)-3-(1-Phenyl-cycloheptanecarbonyloxy)-1-(pyridin-2-ylcarbamoylmethyl)-1-azonia-bicyclo[2.2.2]octane bromide (compound Z) (1 nM) in guinea pig trachea in vitro.

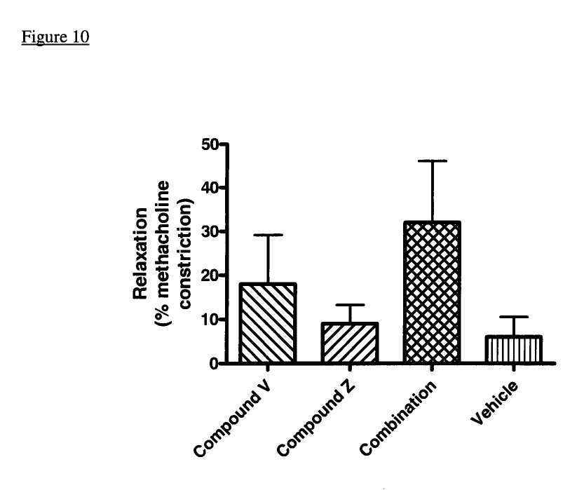

FIG. 10: Percentage relaxation to N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide (compound V) (10 nM), (R)-3-(1-Phenyl-cycloheptanecarbonyloxy)-1-(pyridin-2-ylcarbamoylmethyl)-1-azonia-bicyclo[2.2.2]octane bromide (compound Z) (1 nM) and the combination of N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide (compound V) (10 nM) and (R)-3-(1-Phenyl-cycloheptanecarbonyloxy)-1-(pyridin-2-ylcarbamoylmethyl)-1-azonia-bicyclo[2.2.2]octane bromide (compound Z) (1 nM) in guinea pig trachea in vitro.

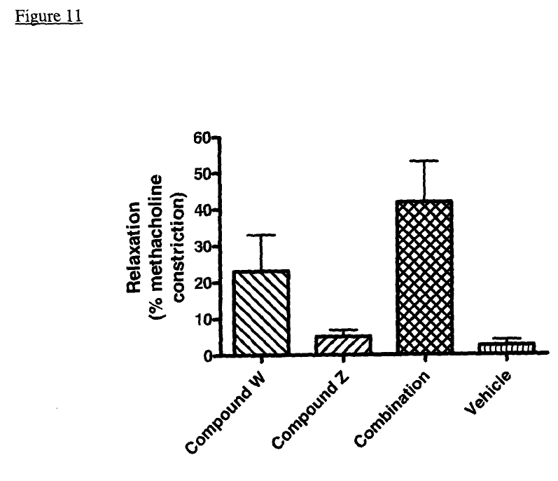

FIG. 11: Percentage relaxation to N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide di-D-mandelate (compound W) (1 nM), (R)-3-(1-Phenyl-cycloheptanecarbonyloxy)-1-(pyridin-2-ylcarbamoylmethyl)-1-azonia-bicyclo[2.2.2]octane bromide (compound Z) (1 nM) and the combination of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide di-D-mandelate salt (compound W) (1 nM) and (R)-3-(1-Phenyl-cycloheptanecarbonyloxy)-1-(pyridin-2-ylcarbamoylmethyl)-1-azonia-bicyclo[2.2.2]octane bromide (compound Z) (1 nM) in guinea pig trachea in vitro.

### PREPARATION OF MUSCARINIC ANTAGONISTS

Muscarinic antagonists according to the present invention may be prepared as follows. Alternative salts to those described herein may be prepared by conventional chemistry using methods analogous to those described.

General Experimental Details for Preparation of Muscarinic Antagonists

Unless otherwise stated the following general conditions were used in the preparation of the Muscarinic Antagonists

All reactions were carried out under an atmosphere of nitrogen unless specified otherwise.

In the examples the NMR spectra were measured on a Varian Unity Inova spectrometer at a proton frequency of either 300 or 400 or 500 MHz, or on a Bruker DRX spectrometer at a proton frequency of 400 or 500 MHz, or on a Bruker Avance spectrometer with a proton frequency of 600 MHz or or on a Bruker Avance DPX 300 spectrometer with a proton frequency of 300 MHz. The MS spectra were measured on either an Agilent 1100 MSD G1946D spectrometer or a Hewlett Packard HP1100 MSD G1946A spectrometer or a Waters Micromass ZQ2000 spectrometer. Names were generated using the Autonom 2000 (version 4.01.305) software supplied by MDL. XRPD data were collected using either a PANalytical CubiX PRO machine or a PANalytical X-Pert machine.

### X-Ray Powder Diffraction—XRPD—PANalytical CubiX PRO

Data was collected with a PANalytical CubiX PRO machine in θ-θ configuration over the scan range 2° to 40° 2θ with 100-second exposure per 0.02° increment. The X-rays were generated by a copper long-fine focus tube operated at 45 kV and 40 mA. The wavelength of the copper X-rays was 1.5418 Å. The Data was collected on zero background holders on which ˜2 mg of the compound was placed. The holder was made from a single crystal of silicon, which had been cut along a non-diffracting plane and then polished on an optically flat finish. The X-rays incident upon this surface were negated by Bragg extinction.

### X-Ray Powder Diffraction—PANalytical X-Pert

Data was collected using a PANalytical X-Pert machine in 2θ-θ configuration over the scan range 2° to 40° 2θ with 100-second exposure per 0.02° increment. The X-rays were generated by a copper long-fine focus tube operated at 45 kV and 40 mA. The wavelengths of the copper X-rays was 1.5418A. The Data was collected on zero background holders on which ˜2 mg of the compound was placed. The holder was made from a single crystal of silicon, which had been cut along a non-diffracting plane and then polished on an optically flat finish. The X-rays incident upon this surface were negated by Bragg extinction.

Differential Scanning calorimetry (DSC) thermograms were measured using a TA Instruments Q1000 DSC Differential Scanning calorimeter, with aluminum pans and pierced lids. The sample weights varied between 0.5 to 5 mg. The procedure was carried out under a flow of nitrogen gas (50 mL/min) and the temperature studied from 25 to 300° C. at a constant rate of temperature increase of 10° C. per minute.

Thermogravimetric Analysis (TGA) thermograms were measured using a TA Instruments Q500 TGA Thermogravimetric Analyser, with platinum pans. The sample weights varied between 1 and 5 mg. The procedure was carried out under a flow of nitrogen gas (60 mL/min) and the temperature studied from 25 up to 200-300° C. at a constant rate of temperature increase of 10° C. per minute.

Gravimetric Vapour Sorption (GVS) profiles were measured using a Surface Measurements Systems Dynamic Vapour Sorption DVS-1, or DVS Advantage GVS instruments. The solid sample ca. 1-5 mg was placed into a glass or wire mesh vessel and the weight of the sample was recorded during a dual cycle step method (40 to 90 to 0 to 90 to 0% relative humidity (RH), in steps of 10% RH).

Abbreviations used in the experimental section:

Aq=aqueous
DCE=1,2-dichloroethane
DCM=dichloromethane
DMF=dimethylformamide

### DMSO=Dimethylsulfoxide

EtOAc=ethyl acetate
EtOH=ethanol
DSC=Differential Scanning calorimeter
GVS=Gravimetric vapour sorption
TGA=Thermogravimetric analysis

### XRPD=X-Ray Powder Diffraction

HATU=O-(7-Azabenzotriazol-1-yl)-N,N,N′N′-tetramethyluronium hexafluorophospahte

### MeCN—Acetonitrile

MeOH=methanol

### RT=Room Temperature

Rt=retention time
THF=tetrahydrofuran
Satd=saturated

Muscarinic antagonists, and the intermediates used in their preparation, described herein have been given the IUPAC names generated by the Beilstein Autonom 2000 naming package, as supplied by MDL Information Systems Inc., based on the structures depicted in the examples, and stereochemistry assigned according to the Cahn-Ingold-Prelog is system.

### EXAMPLE 1

### (R)-3-(1-Phenyl-cycloheptanecarbonyloxy)-1-(pyrazin-2-ylcarbamoylmethyl)-1-azonia-bicyclo[2.2.2]octane bromide

### a) 1-Phenyl-cycloheptanol

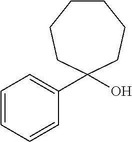

To magnesium (1.2 g) in anhydrous tetrahydrofuran (60 mL) under an environment of nitrogen was added a crystal of iodine followed by bromobenzene (7.85 g) at such a rate that the reaction maintained a steady reflux. The reaction mixture was stirred for 20 minutes then cycloheptanone (4.48 g) was added with care. After stirring for 10 minutes saturated aqueous ammonium chloride (10 mL) was added and the reaction was partitioned between water (100 mL) and isohexane (100 mL). The organic layer was dried (MgSO₄) and evaporated to afford the sub-titled compound (7.6 g) as an oil.

¹H NMR (299.946 MHz, CDCl₃) δ 7.53-7.47 (m, 2H), 7.36-7.29 (m, 2H), 7.26-7.19 (m, 1H), 2.07 (ddd, 2H), 1.97-1.50 (m, 11H).

### b) 1-Methoxy-1-phenyl-cycloheptane

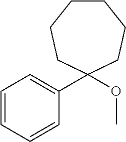

1-Phenyl-cycloheptanol (Example 1a) (7.6 g) was dissolved in tetrahydrofuran (100 mL) and sodium hydride (60% in oil, 2.0 g) added. The reaction was stirred at 60° C. for 5 minutes and iodomethane (7.1 g) added. The mixture was maintained at 60° C. overnight and then further quantities of sodium hydride (60% in oil, 2.0 g) and iodomethane (7.1 g) were added and the reaction was refluxed for 70 hours. The reaction mixture was partitioned between water (100 mL) and isohexane (100 mL) and the organic layer separated, dried (MgSO₄) and evaporated to afford the sub-titled compound (11.31 g).

¹H NMR (300 MHz, CDCl₃) δ 7.43-7.37 (m, 2H), 7.37-7.30 (m, 2H), 7.24-7.19 (m, 1H), 2.98 (s, 3H), 2.12-1.88 (m, 4H), 1.88-1.45 (m, 8H).

### c) 1-Phenyl-cycloheptanecarboxylic acid

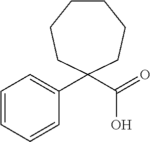

Potassium (2.62 g) and sodium (0.52 g) were heated together at 120° C. in mineral oil under an environment of nitrogen for 30 minutes and then cooled to room temperature. The oil was removed and replaced with ether (100 mL) and 1-methoxy-1-phenyl-cycloheptane (Example 1b) (4.9 g) was added and the reaction was stirred under nitrogen overnight at room temperature. The reaction was cooled to −78° C. and solid carbon dioxide (˜20 g) was added with stirring. The reaction was allowed to warm to room temperature and water (150 mL) was added carefully under an environment of nitrogen. The aqueous layer was separated, neutralised with concentrated hydrochloric acid and extracted with diethyl ether (150 mL). The organic layer was dried (MgSO₄) and evaporated afford to the sub-titled compound (4.15 g) as an oil.

¹H NMR (300 MHz, CDCl₃) δ 7.40-7.20 (m, 5H), 2.49-2.35 (m, 2H), 2.16-2.03 (m, 2H), 1.76-1.47 (m, 8H).

### d) 1-Phenyl-cycloheptanecarboxylic acid methyl ester

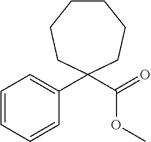

1-Phenyl-cycloheptanecarboxylic acid (Example 1c) (4.15 g) was refluxed in methanol (150 mL) and concentrated hydrochloric acid (5 mL) for 24 hours. The solvent was evaporated and the residue was dissolved in ether (100 mL) which was washed with water (100 mL), saturated sodium bicarbonate (50 mL) and water (100 mL), dried (MgSO₄) and evaporated to afford the sub-titled compound (3.5 g) as an oil.

¹H NMR (300 MHz, CDCl₃) δ 7.37-7.18 (m, 5H), 3.63 (s, 3H), 2.47-2.35 (m, 2H), 2.08-1.97 (m, 2H), 1.70-1.48 (m, 8H).

### e) 1-Phenyl-cycloheptanecarboxylic acid (R)-(1-aza-bicyclo[2.2.2]oct-3-yl)ester

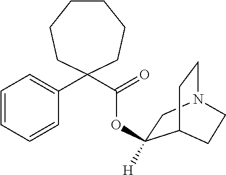

1-Phenyl-cycloheptanecarboxylic acid methyl ester (Example 1d) (1.0 g) and (R)-quinuclidin-3-ol (0.39 g) were refluxed in heptane (50 mL) containing sodium (˜5 mg) in a Dean and Stark apparatus for 24 hours. Heptane (20 mL) was replaced with toluene (20 mL) and the reflux was continued for 3 days. The reaction was partitioned between water (50 mL) and ether (50 mL) and the ether layer was separated, dried (MgSO₄) and evaporated. The crude product was purified by column chromatography on silica eluting with ethyl acetate/triethylamine (99/1) to afford the titled compound as an oil (0.83 g).

### m/e 328 [M+H]⁺

1H NMR (300 MHz, CDCl₃) δ 7.35-7.27 (m, 4H), 7.23-7.16 (m, 1H), 4.78-4.71 (m, 1H), 3.12 (ddd, 1H), 2.79-2.32 (m, 7H), 2.16-1.98 (m, 2H), 1.91-1.80 (m, 1H), 1.70-1.34 (m, 12H).

### f) 2-Bromo-N-pyrazin-2-yl-acetamide

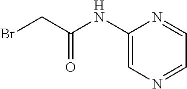

To a stirred suspension of pyrazin-2-ylamine (1.878 g) and potassium carbonate (8.19 g) in dichloromethane (25 mL) was added 2-bromoacetyl bromide (1.72 mL). The reaction was stirred overnight and then washed with water (2×50 mL). The organic layer was dried (MgSO₄) and concentrated to afford the sub-titled compound as a solid (0.700 g).

¹H NMR (400 MHz, CDCl₃) δ 9.51 (d, 1H), 8.63 (s, 1H), 8.42 (d, 1H), 8.30 (dd, 1H), 4.06 (s, 2H).

### EXAMPLE 1

### (R)-3-(1-Phenyl-cycloheptanecarbonyloxy)-1-(pyrazin-2-ylcarbamoylmethyl)-1-azonia-bicyclo[2.2.2]octane bromide Crystalline Form A

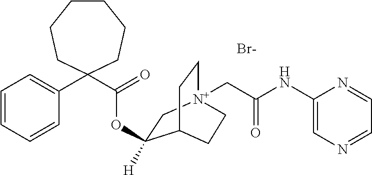

1-Phenyl-cycloheptanecarboxylic acid (R)-(1-aza-bicyclo[2.2.2]oct-3-yl)ester (Example 1e) (0.200 g) and 2-bromo-N-pyrazin-2-yl-acetamide (Example 10 (0.132 g) were dissolved in acetonitrile (1 mL) and left to stand overnight. The resulting solid was filtered and washed with acetonitrile (2×1 mL), and diethyl ether (3 mL). The dried solid was recrystallised from acetone (15 mL) and diethyl ether (10 mL) to afford the titled compound (0.240 g).

m/e 463 [M]⁺

¹H NMR (400 MHz, DMSO-D₆) δ 11.37 (s, 1H), 9.28 (s, 1H), 8.50-8.46 (m, 2H), 7.39-7.30 (m, 4H), 7.27-7.21 (m, 1H), 5.16-5.08 (m, 1H), 4.33 (s, 2H), 4.17-4.07 (m, 1H), 3.69-3.56 (m, 4H), 3.48-3.38 (m, 1H), 2.44-2.26 (m, 3H), 2.25-2.04 (m, 2H), 2.03-1.87 (m, 3H), 1.85-1.71 (m, 1H), 1.68-1.45 (m, 8H).

### Analysis of Example 1

### (R)-3-(1-Phenyl-cycloheptanecarbonyloxy)-1-(pyrazin-2-ylcarbamoylmethyl)-1-azonia-bicyclo[2.2.2]octane bromide Crystalline Form A

A sample of crystalline Example 1 Crystalline Form A obtained by the procedure described above was analysed by XRPD (PANalytical X'Pert or CubiX system), DSC and TGA.

The melting temperature of Example 1 bromide Form A as determined by DSC was found to be 202° C. (onset) (±2° C.). Weight loss observed prior to melting by TGA was 2.7%. GVS determination gave a 3% weight increase (% w/w) at 80% RH (±0.2%).

An XRPD spectrum of Example 1 bromide Form A is presented in FIG. 1.

### EXAMPLE 2

### (R)-3-(1-Phenyl-cycloheptanecarbonyloxy)-1-(pyrazin-2-ylcarbamoylmethyl)-1-azonia-bicyclo[2.2.2]octane chloride

### a) 2-Chloro-N-pyrazin-2-yl-acetamide

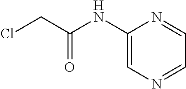

To a stirred suspension of pyrazin-2-ylamine (4.6 g) and potassium carbonate (20.05 g) in dichloromethane (50 mL) was added 2-chloroacetyl chloride (3.85 mL). The reaction was stirred overnight and then washed with water (2×50 mL). The organic layer was dried (MgSO₄) and concentrated to give a solid which was purified by column chromatography on Silica eluting with ethyl acetate/isohexane (5:95) to afford the sub-titled compound as a white solid (2.2 g).

m/e 172 [M+H]⁺

¹H NMR (400 MHz, DMSO-D₆) δ 11.12 (s, 1H), 9.31 (d, 1H), 8.44 (dd, 1H), 8.41 (d, 1H), 4.40 (s, 2H).

### EXAMPLE 2

### (R)-3-(1-Phenyl-cycloheptanecarbonyloxy)-1-(pyrazin-2-ylcarbamoylmethyl)-1-azonia-bicyclo[2.2.2]octane chloride Crystalline Form A

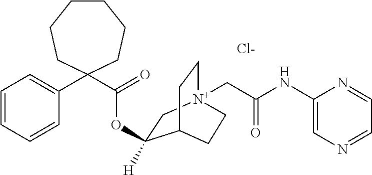

1-Phenyl-cycloheptanecarboxylic acid (R)-(1-aza-bicyclo[2.2.2]oct-3-yl) ester (Example 1e) (0.55 g) and 2-chloro-N-pyrazin-2-yl-acetamide (Example 2a) (0.288 g) were stirred in acetonitrile (4 mL) overnight. Further acetonitrile (14 mL) was added and the mixture stirred for 2 hours. The solid was collected by filtration and washed with diethyl ether (4×10 mL) to afford the titled compound as a solid (0.735 g).

m/e 463 [M]⁺

¹H NMR (400 MHz, DMSO-D₆) δ 11.52 (s, 1H), 9.28 (s, 1H), 8.49-8.45 (m, 2H), 7.38-7.31 (m, 4H), 7.26-7.21 (m, 1H), 5.15-5.10 (m, 1H), 4.44 (d, 1H), 4.40 (d, 1H), 4.14 (ddd, 1H), 3.73-3.59 (m, 4H), 3.48-3.38 (m, 1H), 2.42-2.29 (m, 2H), 2.23-2.12 (m, 2H), 2.04-1.87 (m, 1H), 1.83-1.73 (m, 3H), 1.71-1.45 (m, 9H).

### Analysis of Example 2

(R)-3-(1-Phenyl-cycloheptanecarbonyloxy)-1-(pyrazin-2-ylcarbamoylmethyl)-1-azonia-bicyclo[2.2.2]octane chloride Crystalline Form A

A sample of crystalline Example 2 Crystalline Form A obtained by the procedure described above was analysed by XRPD (PANalytical X′Pert or CubiX system), DSC and TGA.

The melting temperature of Example 2 chloride Form A as determined by DSC was found to be 215° C. (onset) (±2° C.). GVS determination gave a 9% weight increase (% w/w) at 80% RH (±0.2%).

An XRPD spectrum of Example 2 chloride Form A is presented in FIG. 2.

### EXAMPLE 3

### (R)-3-(1-Phenyl-cycloheptanecarbonyloxy)-1-(pyridin-2-ylcarbamoylmethyl)-1-azonia-bicyclo[2.2.2]octane chloride

### a) Cycloheptyl-phenyl-methanone

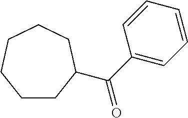

Phenylmagnesium bromide (3.0M solution in diethyl ether) (271 mL), was added dropwise to a stirred (overhead stirrer) solution of cycloheptanecarbonitrile (50 g) in 229 mL diethyl ether under nitrogen at such a rate as to maintain gentle reflux. The reaction mixture was then heated at reflux for 3 hours. TLC indicated no starting material present in the reaction mixture. The reaction mixture was allowed to cool to room temperature and stood under nitrogen overnight. The reaction mixture was cooled to 0° C. and treated dropwise with 102 mL 4N HCl(aq) keeping the temperature below 20° C. 4N sulfuric acid (203 mL) was added dropwise rapidly to start with and then more quickly towards the end. The ice bath was removed and the diethyl ether was distilled off. The reaction mixture was heated at 80-90° C. for 3.5 hours then allowed to cool to room temperature and stood overnight. The mixture was diluted with ether (approx 450 mL) and water (100 mL). The layers were separated and the aqueous layer was extracted with ether (2×400 mL). The organic layers were combined and washed with saturated aqueous sodium hydrogen carbonate (600 mL) and brine (600 mL), dried over magnesium sulphate, filtered and evaporated to give the sub-titled compound as an orange liquid (86.5 g).

¹H NMR (300 MHz, CDCl₃) δ 7.96-7.91 (d, 2H), 7.54-7.49 (m, 1H), 7.48-7.40 (t, 2H), 3.48-3.37 (m, 1H), 1.98-1.88 (m, 2H), 1.85-1.44 (m, 10H).

### b) (1-Chloro-cycloheptyl)-phenyl-methanone

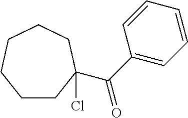

Sulfuryl chloride (210 mL) was added dropwise to neat cycloheptyl-phenyl-methanone (Example 3a) (86.5 g) at 0° C. over approximately 1 hour. Gas evolution and an exotherm were observed. The internal temperature was kept below 15° C. during the addition and the evolved gas was scrubbed by passing through a 10.2M aqueous solution of NaOH. The reaction mixture was heated to reflux overnight. TLC indicated no starting material remained. The reaction mixture was cooled to 0° C. and poured slowly onto ice (1 L) with stirring. The layers were separated and the aqueous layer was extracted with ether (2×400 mL). The combined organic layers were washed with water (600 mL), saturated aqueous sodium hydrogen carbonate (600 mL), and brine (600 mL), dried over magnesium sulphate, filtered and evaporate to give the sub-titled compound as a brown oil (100 g).

¹H NMR (400 MHz, CDCl₃) δ 8.10-8.06 (d, 2H), 7.52-7.46 (t, 1H), 7.44-7.36 (t, 2H), 2.50 (ddd, 2H), 2.29 (ddd, 2H), 1.84-1.73 (m, 2H), 1.68-1.58 (m, 2H), 1.58-1.43 (m, 4H).

### c) 1-Phenyl-cycloheptanecarboxylic acid

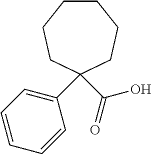

A solution of (1-chloro-cycloheptyl)-phenyl-methanone (Example 3b) (100 g) in 750 mL dioxane was treated dropwise rapidly with a cloudy solution of silver nitrate (137 g) in water (85 mL) causing a precipitate to form. The reaction mixture was heated to 75° C. for 4.5 hours. TLC showed no starting material remaining. The reaction mixture was cooled to room temperature then filtered and concentrated to approximately 200 mL. Water (200 mL) and ether (300 mL) were added and the layers separated. The aqueous layer was extracted with ether (2×250 mL). The combined organic layers were extracted with 10% aqueous sodium carbonate (3×250 mL). The combined basic extracts were heated up to 90° C. over 40 minutes and then cooled to room temperature and acidified with concentrated HCl (aq). The resulting brown solid was filtered off, washed with water (×2) and dried under vacuum at 50° C. Crystallisation from hot ethanol (40 mL) gave the sub-titled compound as pale brown crystals (9.83 g).

¹H NMR (400 MHz, CD₃OD) δ 7.36-7.26 (m, 4H), 7.21-7.15 (m, 1H), 2.43-2.35 (m, 2H), 2.07-1.98 (m, 2H), 1.70-1.53 (m, 8H).

### d) 1-Phenyl-cycloheptanecarboxylic acid methyl ester

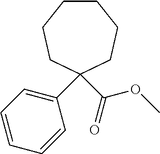

A 2.0 M solution of trimethylsilyl diazomethane (29.2 mL) was added dropwise to a solution of 1-phenyl-cycloheptanecarboxylic acid (Example 3c) (9.8 g) in methanol (85 mL) and toluene (300 mL) under an atmosphere of nitrogen. TLC after 45 minutes showed no starting material present. The reaction mixture was concentrated under vacuum and the crude product was purified by column chromotography eluting with 0-10% ethyl acetate/cyclohexane. The relevant fractions were combined to give the product as a pale yellow oil (9.25 g).

¹H NMR (300 MHz, CD₃OD) δ 7.32-7.24 (m, 4H), 7.21-7.12 (m, 1H), 3.60 (s, 3H), 2.43-2.32 (m, 2H), 2.07-1.96 (m, 2H), 1.65-1.58 (m, 8H).

### e) 1-Phenyl-cycloheptanecarboxylic acid (R)-(1-aza-bicyclo[2.2.2]oct-3-yl) ester

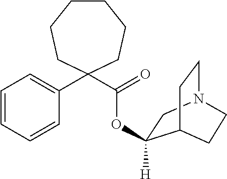

A solution of (R)-(3)-quinuclidinol (10.13 g) and 1-phenyl-cycloheptanecarboxylic acid methyl ester (Example 3d) (9.25 g) in toluene (90 mL) was heated to reflux with a Dean-Stark trap for 30 min. The reaction mixture was allowed to cool to room temperature and the trap was removed. Sodium hydride (60% dispersion in mineral oil) (3.19 g) was added portionwise under nitrogen and the reaction mixture was heated to reflux overnight under nitrogen. TLC showed no starting material remaining. The reaction mixture was cooled in an ice bath and diluted with ethyl acetate (200 mL) and water (200 mL). The mixture was filtered and the layers separated. The aqueous layer was extracted with ethyl acetate (2×250 mL) and the combined organic layers were washed with brine, dried over magnesium sulfate and evaporated to give the crude product which was purified by silica gel chromatography eluting with EtOAc containing 1% triethylamine. The relevant fractions were combined and evaporated to give the sub-titled compound as a colourless oil (7.63 g).

¹H NMR (400 MHz, CD₃OD) δ 7.34-7.28 (m, 4H), 7.23-7.17 (m, 1H), 4.80-4.75 (m, 1H), 3.12 (ddd, 1H), 2.75-2.65 (m, 3H), 2.53-2.37 (m, 4H), 2.14-2.06 (m, 2H), 1.88-1.85 (m, 1H), 1.69-1.54 (m, 10H), 1.54-1.42 (m, 1H), 1.35-1.24 (m, 1H).

### f) 2-Chloro-N-pyridin-2-yl-acetamide

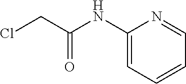

A solution of 2-amino-pyridine (1.0 g) in dry dichloromethane (10.6 mL) under nitrogen at 0° C. was treated with triethylamine (1.63 mL) followed by slow addition of chloroacetyl chloride (0.93 mL). The reaction mixture was allowed to warm up to room temperature. After 2 hours, the mixture was partitioned between dichloromethane and water. The phases were separated and the aqueous layer was extracted with dichloromethane (×2). The combined organic layer was washed with brine, dried over magnesium sulphate, filtered and concentrated to give the crude product which was purified by silica gel chromatography eluting with 0-30% ethyl acetate/cyclohexane. The relevant fractions were combined and evaporated to give the title compound (1.43 g) as a pink solid. Further purification was achieved by trituration with 40-60 petroleum ether to give 1.15 g of the desired product. Crystallisation of a 0.94 g portion of the material from refluxing acetonitrile (2.4 mL) gave the sub-titled compound as a pink solid (0.73 g).

¹H NMR (400 MHz, CDCl₃): δ 8.96 (s, 1H), 8.32 (ddd, 1H), 8.21 (d, 1H), 7.76 (ddd, 1H), 7.12 (ddd, 1H), 4.20 (s, 2H).

### EXAMPLE 3

### (R)-3-(1-Phenyl-cycloheptanecarbonyloxy)-1-(pyridin-2-ylcarbamoylmethyl)-1-azonia-bicyclo[2.2.2]octane chloride Crystalline Form A

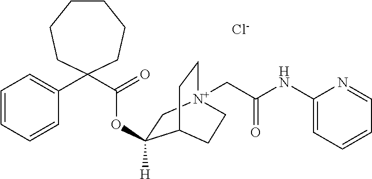

A solution of 1-phenyl-cycloheptanecarboxylic acid (R)-(1-aza-bicyclo[2.2.2]oct-3-yl) ester (Example 3e) (254 mg) in acetonitrile (5 mL) was treated with 2-chloro-N-pyridin-2-yl-acetamide (Example 3f) (46 mg) and the resulting yellow solution was stirred at room temperature overnight during which a solid precipitated. The reaction mixture was treated with a couple of mLs of ether and the solid was filtered off, washed with ether and dried under vacuum to give the title compound (217 mg) as an off-white solid. Purification was achieved by crystallisation from refluxing acetonitrile (20 mL) to give 98 mg of the title compound as a white crystalline solid.

m/e 462 [M]⁺

¹H NMR (400 MHz, DMSO-D₆): δ 11.09 (s, 1H), 8.34-8.32 (d, 1H), 7.97 (d, 1H), 7.85-7.79 (t, 1H), 7.33-7.25 (m, 4H), 7.21-7.13 (m, 2H), 5.07 (m, 1H), 4.29 (s, 2H), 4.07 (ddd, 1H), 3.65-3.51 (m, 4H), 3.41-3.29 (m, 1H), 2.36-2.23 (m, 2H), 2.17-2.04 (m, 2H), 1.99-1.81 (m, 3H), 1.78-1.66 (m, 1H), 1.77-1.19 (m, 9H).

### Analysis of Example 3

### (R)-3-(1-Phenyl-cycloheptanecarbonyloxy)-1-(pyridin-2-ylcarbamoylmethyl)-1-azonia-bicyclo[2.2.2]octane chloride Crystalline Form A

A sample of crystalline Example 3 Crystalline Form A obtained by the procedure described above was analysed by XRPD (PANalytical X'Pert system), DSC and TGA.

The melting temperature of Example 3 chloride Form A as determined by DSC was found to be 239° C. (onset) (±2° C.). Weight loss observed prior to melting by TGA was negligible. GVS determination gave a negligible weight increase (% w/w) at 80% RH (±0.2%).

An XRPD spectrum of Example 3 chloride Form A is presented in FIG. 3.

### EXAMPLE 4

### (R)-3-(1-Phenyl-cycloheptanecarbonyloxy)-1-(pyridin-2-ylcarbamoylmethyl)-1-azonia-bicyclo[2.2.2]octane bromide

### a) 2-Bromo-N-pyridin-2-yl-acetamide

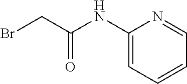

To a solution of 2-aminopyridine (48.8 mmol) in anhydrous THF (98 mL) at room temperature was added Et₃N (58.6 mmol) and bromoacetyl bromide (58.6 mmol) dropwise. The mixture was stirred overnight and quenched with sat. NaHCO_{3 (aq)}. EtOAc was added to the mixture and the layers separated. The aqueous phase was extracted with EtOAc and the combined organics dried (MgSO₄) and concentrated in vacuo to a brown solid. Purification by flash silica gel chromatography eluting with 1-2% MeOH/dichloromethane gave the sub-titled compounds as a yellow solid (1.14 g).

¹H NMR (400 MHz, CDCl₃): δ 8.75 (s, 1H), 8.26 (ddd, 1H), 8.10 (d, 1H), 7.67 (ddd, 1H), 7.03 (ddd, 1H), 3.94 (s, 2H).

### EXAMPLE 4

### (R)-3-(1-Phenyl-cycloheptanecarbonyloxy)-1-(pyridin-2-ylcarbamoylmethyl)-1-azonia-bicyclo[2.2.2]octane bromide Crystalline Form A

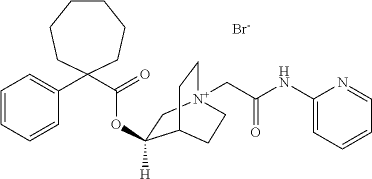

1-Phenyl-cycloheptanecarboxylic acid (R)-(1-aza-bicyclo[2.2.2]oct-3-yl) ester (Example 3e) (0.79 mmol) and 2-bromo-N-pyridin-2-yl-acetamide (Example 4a) (0.87 mmol) were stirred together in anhydrous MeCN at room temperature for 2.5 days. The reaction mixture was concentrated in vacuo and the yellow solid purified by flash silica gel column chromatography eluting with 2-8% MeOH/dichloromethane to give a tan solid. The solid was dissolved up in refluxing MeCN and the solution was allowed to cool down to room temperature. The resulting crystals were filtered off and washed with a small quantity of cold MeCN to give the title compound (211 mg) as a white crystalline solid.

m/e 462 [M]⁺

¹H NMR (400 MHz, DMSO-D₆): δ 11.02 (s, 1H), 8.33 (ddd, 1H), 7.97 (d, 1H), 7.86-7.80 (m, 1H), 7.32-7.25 (m, 4H), 7.23-7.12 (m, 2H), 5.09-5.04 (m, 1H), 4.23 (s, 2H), 4.06 (ddd, 1H), 3.63-3.49 (m, 4H), 3.41-3.29 (m, 1H), 2.37-2.22 (m, 2H), 2.17-2.04 (m, 2H), 1.98-1.83 (m, 3H), 1.78-1.66 (m, 1H), 1.65-1.39 (m, 9H).

### Analysis of Example 4

### (R)-3-(1-Phenyl-cycloheptanecarbonyloxy)-1-(pyridin-2-ylcarbamoylmethyl)-1-azonia-bicyclo[2.2.2]octane bromide Crystalline Form A

A sample of crystalline Example 4 Crystalline Form A obtained by the procedure described above was analysed by XRPD (PANalytical X′Pert system), DSC and TGA.

The melting temperature of Example 4 bromide Form A as determined by DSC was found to be 230° C. (onset) (±2° C.). Weight loss observed prior to melting by TGA was negligible. GVS determination gave a negligible weight increase (% w/w) at 80% RH (±0.2%).

An XRPD spectrum of Example 4 bromide Form A is presented in FIG. 4.

### EXAMPLE 5

### (R)-3-(1-Phenyl-cycloheptanecarbonyloxy)-1-(pyrazin-2-ylcarbamoylmethyl)-1-azonia-bicyclo[2.2.2]octane 1-hydroxy-naphthalene-2-sulfonate Crystalline Form A

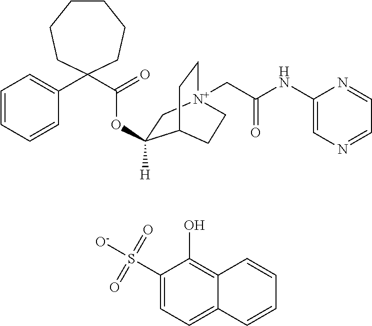

(R)-3-(1-Phenyl-cycloheptanecarbonyloxy)-1-(pyrazin-2-ylcarbamoylmethyl)-1-azonia-bicyclo[2.2.2]octane chloride (Example 2) (100 mg) and 1-hydroxynaphthalene-2-sulfonic acid potassium salt (200 mg) were partitioned between water (10 mL) and dichloromethane (25 mL) in a separating funnel. The dichloromethane was separated and washed with water (10 mL) and the organic layer was dried, evaporated to a solid which was recrystallised from acetonitrile to afford the titled compound as a solid (97 mg).

m/e 463 [M]⁺

¹H NMR (400 MHz, DMSO-D₆) δ 11.61 (s, 1H), 11.36 (s, 1H), 9.28 (s, 1H), 8.49-8.45 (m, 2H), 8.17 (d, 1H), 7.81 (d, 1H), 7.56-7.46 (m, 3H), 7.38-7.29 (m, 5H), 7.27-7.21 (m, 1H), 5.16-5.09 (m, 1H), 4.30 (s, 2H), 4.16-4.07 (m, 1H), 3.68-3.54 (m, 4H), 3.48-3.35 (m, 1H), 2.42-2.27 (m, 2H), 2.25-2.10 (m, 2H), 2.03-1.89 (m, 3H), 1.84-1.71 (m, 1H), 1.66-1.51 (m, 9H).

### Analysis of Example 5

### (R)-3-(1-Phenyl-cycloheptanecarbonyloxy)-1-(pyrazin-2-ylcarbamoylmethyl)-1-azonia-bicyclo[2.2.2]octane 1-hydroxy-naphthalene-2-sulfonate Crystalline Form A

A sample of crystalline Example 5 Crystalline Form A obtained by the procedure described above was analysed by XRPD (PANalytical X'Pert or CubiX system) and DSC.

The melting temperature of Example 5 1-hydroxy-naphthalene-2-sulfonate Form A as determined by DSC was found to be 193° C. (onset) (±2° C.). GVS determination gave a negligible weight increase, near 0.3% (% w/w) at 80% RH (±0.2%).

An XRPD spectrum of Example 5 1-hydroxy-naphthalene-2-sulfonate Form A is presented in FIG. 5.

### EXAMPLE 6

### (R)-3-(1-Phenyl-cycloheptanecarbonyloxy)-1-(pyrazin-2-ylcarbamoylmethyl)-1-azonia-bicyclo[2.2.2]octane 2,5-dichloro-benzenesulfonate

### Crystalline Form A

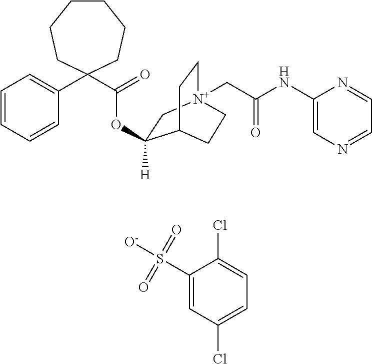

(R)-3-(1-Phenyl-cycloheptanecarbonyloxy)-1-(pyrazin-2-ylcarbamoylmethyl)-1-azonia-bicyclo[2.2.2]octane chloride (Example 2) (100 mg) was suspended between water (10 mL) and dichloromethane (25 mL) in a separating funnel. An aqueous solution of 2,5-dichlorobenzenesulfonic acid sodium salt (0.1M, 8 mL) was added and the mixture shaken. The dichloromethane was separated and washed with water (10 mL) and the organic layer was dried, evaporated to a solid which was recrystallised from acetonitrile/diethyl ether to afford the titled compound (81 mg).

m/e 463 [M]⁺

¹H NMR (400 MHz, DMSO-D₆) δ 11.36 (s, 1H), 9.27 (s, 1H), 8.49-8.44 (m, 2H), 7.83 (d, 1H), 7.43-7.31 (m, 6H), 7.27-7.22 (m, 1H), 5.16-5.09 (m, 1H), 4.36-4.25 (m, 2H), 4.16-4.07 (m, 1H), 3.69-3.55 (m, 4H), 3.48-3.36 (m, 1H), 2.42-2.28 (m, 2H), 2.23-2.10 (m, 2H), 2.03-1.87 (m, 3H), 1.83-1.72 (m, 1H), 1.69-1.46 (m, 9H).

### Analysis of Example 6

### (R)-3-(1-Phenyl-cycloheptanecarbonyloxy)-1-(pyrazin-2-ylcarbamoylmethyl)-1-azonia-bicyclo[2.2.2]octane 2,5-dichloro-benzenesulfonate

### Crystalline Form A

A sample of crystalline Example 6 Crystalline Form A obtained by the procedure described above was analysed by XRPD (PANalytical X′Pert system) and DSC.

The melting temperature of Example 6 2,5-dichloro-benzenesulfonate Form A as determined by DSC was found to be 158° C. (onset) (±2° C.). GVS determination gave a negligible weight increase, near 0.2% (% w/w) at 80% RH (±0.2%).

An XRPD spectrum of Example 6 2,5-dichloro-benzenesulfonate Form A is presented in FIG. 6.

### EXAMPLE 7

### (R)-3-(1-Phenyl-cycloheptanecarbonyloxy)-1-(pyrazin-2-ylcarbamoylmethyl)-1-azonia-bicyclo[2.2.2]octane hemi-naphthalene-1,5-disulfonate Crystalline Form A

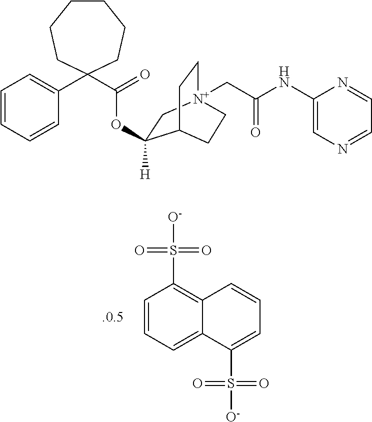

(R)-3-(1-Phenyl-cycloheptanecarbonyloxy)-1-(pyrazin-2-ylcarbamoylmethyl)-1-azonia-bicyclo[2.2.2]octane chloride (Example 2) (100 mg) in dichloromethane (25 mL) was washed with 4×10 mL of an aq. solution of naphthalene-1,5-disulfonic acid disodium salt (made by addition of 2.88 g acid to 1.68 g of sodium bicarbonate in 100 mL). The organic phase was collected and dried (MgSO₄) then concentrated to dryness. The residue was dissolved in acetone (1 mL) and diethyl ether (3 mL) and the solution allowed to crystallise to afford the titled compound (78 mg).

m/e 463 [M]⁺

¹H NMR (400 MHz, DMSO-D₆) δ 11.10 (s, 1H), 9.22 (s, 1H), 8.94 (d, 1H), 8.43 (d, 2H), 7.94 (d, 1H), 7.28-7.38 (m, 5H), 7.17-7.26 (m, 1H), 5.09-5.17 (m, 1H), 4.25-4.32 (m, 2H), 4.05-4.16 (m, 1H), 3.54-3.68 (m, 4H), 3.35-3.50 (m, 1H), 2.96-3.04 (m, 3H), 2.28-2.41 (m, 1H), 2.10-2.26 (m, 1H), 1.90-2.10 (m, 2H), 1.73-1.86 (m, 1H), 1.48-1.72 (m, 9H).

### Analysis of Example 7

### (R)-3-(1-Phenyl-cycloheptanecarbonyloxy)-1-(pyrazin-2-ylcarbamoylmethyl)-1-azonia-bicyclo[2.2.2]octane hemi-naphthalene-1,5-disulfonate Crystalline Form A

A sample of crystalline Example 7 Crystalline Form A obtained by the procedure described above was analysed by XRPD (PANalytical X'Pert or CubiX system) and DSC.

The melting temperature of Example 7 hemi-naphthalene-1,5-disulfonate Form A as determined by DSC was found to be 222° C. (onset) (±2° C.). GVS determination gave a 1.6% weight increase (% w/w) at 80% RH (±0.2%).

An XRPD spectrum of Example 7 hemi-naphthalene-1,5-disulfonate Form A is presented in FIG. 7.

### EXAMPLE 8

### (R)-3-[1-(3-Fluoro-phenyl)-cycloheptanecarbonyloxy]-1-(pyrazin-2-ylcarbamoylmethyl)-1-azonia-bicyclo[2.2.2]octane bromide

### a) 2-But-3-enyl-2-(3-fluoro-phenyl)-hex-5-enoic acid methyl ester

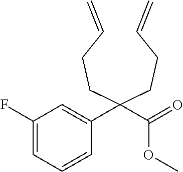

(3-Fluoro-phenyl)-acetic acid methyl ester (4.30 g) was dissolved in tetrahydrofuran (20 mL) and cooled to −78° C. Lithium bis(trimethylsilyl)amide (25.6 mL, 1M THF solution) was added and the solution was stirred for 30 minutes. 4-Bromo-but-1-ene (2.60 mL) was added and the reaction was allowed to warm to room temperature and stirred for an hour. The reaction was again cooled to −78° C. Lithium bis(trimethylsilyl)amide (25.6 mL, 1M THF solution) was added and the solution was stirred for 30 minutes. 4-Bromo-1-butene (2.60 mL) was added and the reaction was allowed to warm to room temperature and stirred for an hour. The reaction was again cooled to −78° C. and further aliquots of Lithium bis(trimethylsilyl)amide (25.6 mL, 1M THF solution) and 4-bromo-1-butene (2.60 mL) were added following the procedure outlined above. After stirring overnight, water (20 mL) was added and the reaction mixture extracted with diethyl ether (2×60 mL). The combined organic extracts were dried with magnesium sulfate and evaporated. The resulting liquid was purified by column chromatography on silica eluting with ethyl acetate/isohexane (1/99) to afford the sub-titled compound (5.0 g).

m/e 277 [M+H]⁺

### b) 1-(3-Fluoro-phenyl)-cyclohept-4-enecarboxylic acid methyl ester

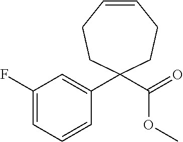

To 2-but-3-enyl-2-(3-fluoro-phenyl)-hex-5-enoic acid methyl ester (Example 8a) (5.0 g) in dichloromethane (100 mL) was added Grubbs Catalyst (2nd Generation, Sigma-Aldrich Company Ltd) (0.05 g). The mixture was warmed to reflux under nitrogen. After 20 hours the reaction was cooled to room temperature, evaporated to an oil and purified by column chromatography on silica eluting with ethyl acetate/isohexane (5/95) to yield an oil. Analysis of the product showed that significant amounts of starting material was present in the mixture so the mixture was subjected to a repetition of the reaction conditions and purification as above to afford the subtitled compound as a coloured oil (3.60 g).

m/e 249 [M+H]⁺

### c) 1-(3-Fluoro-phenyl)-cycloheptanecarboxylic acid methyl ester

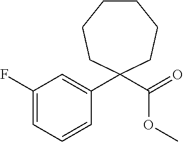

1-(3-Fluoro-phenyl)-cyclohept-4-enecarboxylic acid methyl ester (Example 8b) (1.09 g) was dissolved in methanol (20 mL), palladium on carbon (50 mg) added and mixture stirred under 4 atm of hydrogen overnight. The solution was filtered and evaporated to afford the sub-titled compound (1.09 g).

m/e 251 [M+H]⁺

### d) 1-(3-Fluoro-phenyl)-cycloheptanecarboxylic acid (R)-(1-aza-bicyclo[2.2.2]oct-3-yl) ester

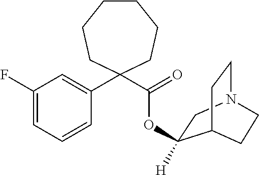

1-(3-Fluoro-phenyl)-cycloheptanecarboxylic acid methyl ester (Example 8c) (0.280 g) was dissolved in toluene (100 mL) and (R)-quinuclidin-3-ol (0.320 g) was added. Toluene (10 mL) was distilled off in a Dean and Stark apparatus and after cooling sodium hydride (10 mg) was added. The reaction was refluxed in a Dean and Stark apparatus for 4 hours after which time an extra amount of sodium hydride (10 mg) was added and the reaction was refluxed for a further for 4 hours. After allowing to cool to room temperature, the toluene was washed with water, dried and evaporated. The residue was purified by column chromatography eluting with ethyl acetate/isohexane/triethylamine (50/50/1) then ethyl acetate/triethylamine (99/1) to afford the sub-titled compound (0.200 g).

m/e 346 [M+H]⁺

¹H NMR (400 MHz, CDCl₃) δ 7.26 (td, 1H), 7.10-7.07 (m, 1H), 7.04 (dd, 1H), 6.90 (ddd, 1H), 4.78-4.73 (m, 1H), 3.14 (ddd, 1H), 2.79-2.66 (m, 3H), 2.66-2.56 (m, 1H), 2.53-2.46 (m, 1H), 2.46-2.36 (m, 2H), 2.13-1.99 (m, 2H), 1.90-1.85 (m, 1H), 1.73-1.40 (m, 11H), 1.29-1.18 (m, 1H).

### EXAMPLE 8

### (R)-3-[1-(3-Fluoro-phenyl)-cycloheptanecarbonyloxy]-1-(pyrazin-2-ylcarbamoylmethyl)-1-azonia-bicyclo[2.2.2]octane bromide

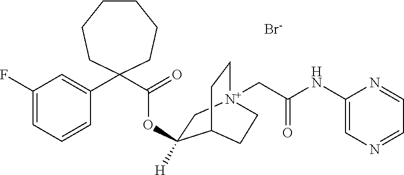

1-(3-Fluoro-phenyl)-cycloheptanecarboxylic acid (R)-(1-aza-bicyclo[2.2.2]oct-3-yl) ester (Example 8d) (0.100 g) was dissolved in acetonitrile (8 mL) and 2-bromo-N-pyrazin-2-yl-acetamide (Example 10 (0.05 g) was added. The reaction was stirred for 3 days, diluted with diethyl ether (8 mL), stirred for a further 10 minutes, the resulting solid was filtered and washed with diethyl ether (3×8 mL) to afford a solid which was recrystallised from hot butanone (8 mL) to afford the titled compound as a solid (0.081 g).

m/e 481 [M⁺]

¹H NMR (400 MHz, DMSO-D₆) δ 11.42 (s, 1H), 9.28 (s, 1H), 8.49-8.45 (m, 2H), 7.40 (td, 1H), 7.19-7.12 (m, 2H), 7.09 (td, 1H), 5.17-5.10 (m, 1H), 4.40-4.30 (m, 2H), 4.16-4.07 (m, 1H), 3.71-3.57 (m, 4H), 3.52-3.41 (m, 1H), 2.43-2.27 (m, 2H), 2.26-2.19 (m, 1H), 2.19-2.09 (m, 1H), 2.05-1.87 (m, 3H), 1.86-1.76 (m, 1H), 1.71-1.46 (m, 9H).

### EXAMPLE 9

### (R)-3-[1-(3-Fluoro-phenyl)-cycloheptanecarbonyloxy]-1-(isoxazol-3-ylcarbamoylmethyl)-1-azonia-bicyclo[2.2.2]octane bromide

### a) 2-Bromo-N-isoxazol-3-yl-acetamide

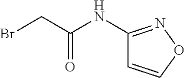

Isoxazol-3-ylamine (1.14 g) was dissolved in dichloromethane (50 mL) and potassium carbonate (3.74 g) was added. Bromoacetyl chloride (1.12 mL) was added slowly with stirring and the suspension was stirred overnight. The reaction was washed with water (2×50 mL), dried and evaporated. The product was recrystallised from dichloromethane/isohexane to afford the sub-titled compound (2.3 g).

¹H NMR (300 MHz, CDCl₃) δ 8.94 (s, 1H), 8.34 (s, 1H), 7.06 (s, 1H), 4.03 (s, 2H).

### EXAMPLE 9

### (R)-3-[1-(3-Fluoro-phenyl)-cycloheptanecarbonyloxy]-1-(isoxazol-3-ylcarbamoylmethyl)-1-azonia-bicyclo[2.2.2]octane bromide

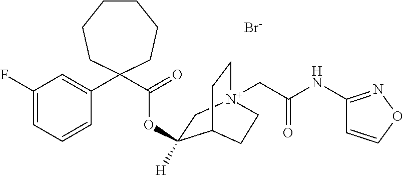

1-(3-Fluoro-phenyl)-cycloheptanecarboxylic acid (R)-(1-aza-bicyclo[2.2.2]oct-3-yl) ester (Example 8d) (50 mg) and 2-bromo-N-isoxazol-3-yl-acetamide (Example 9a) (30 mg) were dissolved in acetonitrile (4 mL) and stirred overnight. The solution was diluted with diethyl ether (12 mL) and stirred overnight. The resulting crystals were filtered off, washed with ether (3×10 mL) and dried to afford the titled compound as a solid (48 mg).

m/e 470 [M⁺]

¹H NMR (400 MHz, DMSO-D₆) δ 11.69 (s, 1H), 8.90 (d, 1H), 7.40 (td, 1H), 7.18-7.07 (m, 3H), 6.91 (d, 1H), 5.16-5.10 (m, 1H), 4.31 (d, 1H), 4.25 (d, 1H), 4.09 (ddd, 1H), 3.68-3.53 (m, 4H), 3.43 (dd, 1H), 2.42-2.27 (m, 2H), 2.25-2.19 (m, 1H), 2.18-2.09 (m, 1H), 2.04-1.88 (m, 3H), 1.85-1.75 (m, 1H), 1.69-1.51 (m, 9H).

### EXAMPLE 10

### (R)-1-[(5-Fluoro-pyridin-2-ylcarbamoyl)-methyl]-3-(1-phenyl-cycloheptanecarbonyloxy)-1-azonia-bicyclo[2.2.2]octane chloride

### a) 2-Chloro-N-(5-fluoro-pyridin-2-yl)-acetamide

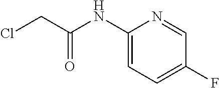

The title compound (0.99 g, 73%, white solid) was prepared according to the method used in Example 3f using 2-amino-5-fluoro-pyridine.

¹H NMR (400 MHz, DMSO-D₆) δ 10.91 (s, 1H), 8.35 (d, 1H), 8.10 (dd, 1H), 7.80-7.74 (m, 1H), 4.34 (s, 2H).

### EXAMPLE 10

### (R)-1-[(5-Fluoro-pyridin-2-ylcarbamoyl)-methyl]-3-(1-phenyl-cycloheptanecarbonyloxy)-1-azonia-bicyclo[2.2.2]octane chloride

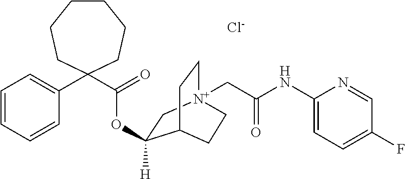

2-Chloro-N-(5-fluoro-pyridin-2-yl)-acetamide (Example 10a) (31 mg) was added to a solution of 1-phenyl-cycloheptanecarboxylic acid (R)-(1-aza-bicyclo[2.2.2]oct-3-yl) ester (Example 3e) (49 mg) in acetonitrile (1 mL). The reaction mixture was stirred at room temperature overnight. Diethyl ether (2 mL) was added to the reaction mixture and the white solid was filtered off, washed several times with diethyl ether and dried under vacuum at 40° C. to give the title compound (49 mg).

m/e 480 [M]⁺

¹H NMR (400 MHz, DMSO-D₆) δ 11.19 (s, 1H), 8.36 (d, 1H), 8.02 (m, 1H), 7.81 (ddd, 1H), 7.33-7.26 (m, 4H), 7.22-7.17 (m, 1H), 5.07 (m, 1H), 4.26 (s, 2H), 4.11-4.03 (m, 1H), 3.64-3.50 (m, 4H), 3.41-3.29 (m, 1H), 2.36-2.23 (m, 2H), 2.17-2.05 (m, 2H), 1.99-1.82 (m, 3H), 1.78-1.65 (m, 1H), 1.70-1.41 (m, 9H).

### EXAMPLE 11

### (R)-1-[(2-Methyl-pyridin-4-ylcarbamoyl)-methyl]-3-(1-phenyl-cycloheptanecarbonyloxy)-1-azonia-bicyclo[2.2.2]octane chloride

### a) 2-Chloro-N-(2-methyl-pyridin-4-yl)-acetamide

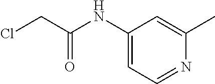

The title compound (1.0 g) was prepared according to the method used in Example 3f using 4-amino-2-methylpyridine.

¹H NMR (400 MHz, DMSO-D₆) δ 10.64 (s, 1H), 8.32 (d, 1H), 7.44 (d, 1H), 7.38-7.35 (m, 1H), 4.30 (s, 2H), 2.42 (s, 3H).

### EXAMPLE 11

### (R)-1-[(2-Methyl-pyridin-4-ylcarbamoyl)-methyl]-3-(1-phenyl-cycloheptanecarbonyloxy)-1-azonia-bicyclo[2.2.2]octane chloride

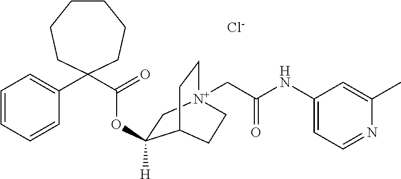

The title compound was prepared using an analogous procedure to that used to prepare Example 10. Further purification was achieved by silica gel chromatography eluting with 0-20% MeOH/dichloromethane to give the title compound as a white solid (57 mg).

m/e 476 [M]⁺

¹H NMR (400 MHz, DMSO-D₆) δ 11.32 (s, 1H), 8.31 (d, 1H), 7.43 (d, 1H), 7.35-7.26 (m, 5H), 7.22-7.16 (m, 1H), 5.09-5.04 (m, 1H), 4.30 (dd, 2H), 4.09-4.01 (m, 1H), 3.64-3.49 (m, 4H), 3.41-3.29 (m, 1H), 2.38 (s, 3H), 2.39-2.23 (m, 2H), 2.17-2.05 (m, 2H), 1.97-1.82 (m, 3H), 1.78-1.65 (m, 1H), 1.65-1.41 (m, 9H).

### EXAMPLE 12

### (R)-3-(1-Phenyl-cycloheptanecarbonyloxy)-1-(pyridin-3-ylcarbamoylmethyl)-1-azonia-bicyclo[2.2.2]octane chloride

### a) 2-Chloro-N-pyridin-3-yl-acetamide

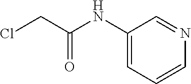

A mixture of 3-aminopyridine (350 mg) and sodium hydroxide (0.6 g) were dissolved in water (8 mL) and the reaction mixture was cooled in an ice bath. Chloroacetyl chloride (1.19 mL) was added dropwise and the reaction mixture was allowed to stir at room temperature overnight. The reaction mixture was extracted with dichloromethane and the organic layer was concentrated and purified by column chromatography, eluting with 0-60% ethyl acetate/cyclohexane to give the title compound (0.10 g) as a white solid.

¹H NMR (400 MHz, DMSO-D₆) δ 10.51 (s, 1H), 8.73 (d, 1H), 8.30 (dd, 1H), 8.03 (ddd, 1H), 7.40-7.35 (m, 1H), 4.30 (s, 2H).

### EXAMPLE 12

### (R)-3-(1-Phenyl-cycloheptanecarbonyloxy)-1-(pyridin-3-ylcarbamoylmethyl)-1-azonia-bicyclo[2.2.2]octane chloride

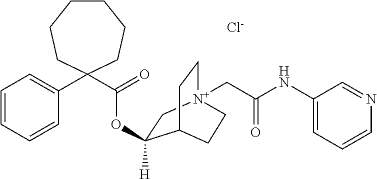

The title compound (78 mg) was prepared by an analogous method to that used in Example 3 using 2-chloro-N-pyridin-3-yl-acetamide in place of 2-bromo-N-pyridin-2-yl-acetamide.

m/e 462 [M]⁺

¹H NMR (400 MHz, DMSO-D₆) δ 11.27 (s, 1H), 8.76 (d, 1H), 8.30 (dd, 1H), 7.98 (ddd, 1H), 7.37 (ddd, 1H), 7.33-7.25 (m, 4H), 7.22-7.15 (m, 1H), 5.07 (d, 1H), 4.28 (dd, 2H), 4.11-4.03 (m, 1H), 3.65-3.50 (m, 4H), 3.41-3.29 (m, 1H), 2.37-2.21 (m, 2H), 2.19-2.05 (m, 2H), 1.97-1.83 (m, 3H), 1.78-1.66 (m, 1H), 1.71-1.27 (m, 9H).

### EXAMPLE 13

### (R)-3-(1-Phenyl-cycloheptanecarbonyloxy)-1-(pyridazin-3-ylcarbamoylmethyl)-1-azonia-bicyclo[2.2.2]octane bromide

### a) 2-Bromo-N-pyridazin-3-yl-acetamide

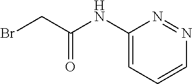

To a suspension of pyridazin-3-ylamine (2.7 g) and diisopropylethylamine (6.3 mL) in dichloromethane (100 mL) at 0° C. was added bromoacetic anhydride (9.0 g) in dichloromethane (10 mL) by dropwise addition. The mixture was stirred at 0° C. for 0.5 hours and then allowed to warm to rt. The resulting suspension was filtered, washed with dichloromethane and dried to afford the sub-titled compound as a solid (2.0 g).

¹H NMR (400 MHz, DMSO-D₆) δ 11.51 (s, 1H), 9.00 (dd, 1H), 8.28 (dd, 1H), 7.74-7.68 (m, 1H), 4.15 (s, 2H).

### EXAMPLE 13

### (R)-3-(1-Phenyl-cycloheptanecarbonyloxy)-1-(pyridazin-3-ylcarbamoylmethyl)-1-azonia-bicyclo[2.2.2]octane bromide

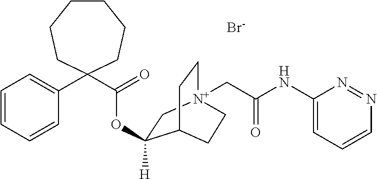

1-Phenyl-cycloheptanecarboxylic acid (R)-(1-aza-bicyclo[2.2.2]oct-3-yl) ester (Example 1e) (0.160 g) and 2-bromo-N-pyridazin-3-yl-acetamide (Example 13a) (0.106 g) were dissolved in acetonitrile (1 mL) and left to stand overnight. The solvents were removed under reduced pressure and the residue purified by chromatography on silica eluting with methanol/dichloromethane (1:9) to afford the titled compound as a solid (180 mg).

m/e 463 [M]⁺

¹H NMR (400 MHz, DMSO-D₆) δ 11.68 (s, 1H), 9.06 (dd, 1H), 8.25 (d, 1H), 7.79 (dd, 1H), 7.39-7.30 (m, 4H), 7.27-7.21 (m, 1H), 5.15-5.10 (m, 1H), 4.34 (s, 2H), 4.16-4.06 (m, 2H), 3.69-3.56 (m, 4H), 3.46-3.36 (m, 1H), 2.43-2.27 (m, 2H), 2.24-2.10 (m, 2H), 2.04-1.89 (m, 3H), 1.84-1.71 (m, 1H), 1.68-1.45 (m, 8H).

### EXAMPLE 14

### (R)-3-(1-Phenyl-cycloheptanecarbonyloxy)-1-(pyridin-3-ylcarbamoylmethyl)-1-azonia-bicyclo[2.2.2]octane hemi-naphthalene-1,5-disulfonate Crystalline Form A

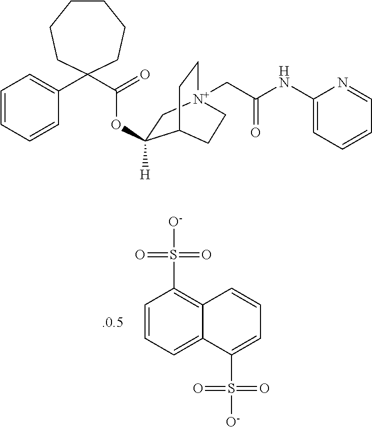

(R)-3-(1-Phenyl-cycloheptanecarbonyloxy)-1-(pyridin-2-ylcarbamoylmethyl)-1-azonia-bicyclo[2.2.2]octane chloride (Example 3) (100 mg) in dichloromethane (4 mL) was shaken with an aqueous solution of naphthalene-1,5-disulfonic acid disodium salt (73 mg in 2 mL of H₂O). The organic phase was collected and the aqueous layer extracted with dichloromethane (4 mL). The combined organic layers were passed through a phase separation cartridge and the resulting solution evaporated to give a colourless oil. The residue was triturated with diethyl ether and the resulting solid collected by filtration, washed with diethyl ether and dried at 50° C. under vacuum. The solid was dissolved in hot acetonitrile (1 mL) and then evaporated to give a foam, which was then dissolved in acetone (2 mL). The mixture was left to stand for 48 h during which crystallization occurred. The resulting crystals were collected by filtration, washed with ice-cooled acetone and then dried at 50° C. under vacuum to afford the titled compound as a white solid (67 mg).

m/e 462 [M]⁺

¹H NMR (400 MHz, DMSO-D₆): δ 11.06 (s, 1H), 8.85-8.88 (d, 1H), 8.40-8.36 (d, 1H), 7.98-8.06 (d, 1H), 7.91-7.94 (dd, 1H), 7.90-7.85 (dd, 1H), 7.42-7.36 (dd, 1H), 7.33-7.25 (m, 4H), 7.21-7.13 (m, 2H), 5.07 (m, 1H), 4.29 (s, 2H), 4.07 (ddd, 1H), 3.65-3.51 (m, 4H), 3.41-3.29 (m, 1H), 2.36-2.23 (m, 2H), 2.17-2.04 (m, 2H), 1.99-1.81 (m, 3H), 1.78-1.66 (m, 1H), 1.77-1.19 (m, 9H).

### Analysis of Example 14

### (R)-3-(1-Phenyl-cycloheptanecarbonyloxy)-1-(pyridin-2-ylcarbamoylmethyl)-1-azonia-bicyclo[2.2.2]octane hemi-naphthalene-1,5-disulfonate Crystalline Form A

A sample of crystalline Example 14 Crystalline Form A obtained by the procedure described above was analysed by XRPD (PANalytical X'Pert or CubiX system) and DSC.

The melting temperature of Example 14 hemi-naphthalene-1,5-disulfonate Form A as determined by DSC was found to be 198° C. (onset) (±2° C.). GVS determination gave a 1% weight increase (% w/w) at 80% RH (±0.3%).

An XRPD spectrum of Example 14 hemi-naphthalene-1,5-disulfonate Form A is presented in FIG. 8.

### Alternative preparation of (R)-3-(1-Phenyl-cycloheptanecarbonyloxy)-1-(pyridine-2-ylcarbamoylmethyl)-1-azoniabicyclo[2.2.2]octane bromide (Example 4)

General Conditions: Unless otherwise stated all reactions were carried out under an inert atmosphere (nitrogen); reagents and solvents were obtained commercially and used as received; reagent grade solvents were used

### (a) Cycloheptanecarboxylic Acid Methyl Ester

Cycloheptanecarboxylic acid (3.75 kg) and methanol (37.50 L) were charged to a reaction vessel and the resultant mixture stirred. Sulfuric Acid (100%, 51.73 g) was charged, the temperature raised to 60° C. and stirring continued for 18 hours. Methanol was removed by distillation under reduced pressure to leave a total volume of 11.25 L. Toluene (37.50 L) was charged and a further 15 L of solvent removed by distillation under reduced pressure. Analysis by ¹H NMR spectroscopy was carried out to confirm that methanol was no longer present in the solution. The mixture was allowed to cool to ambient temperature and diluted with toluene (7.50 L). Saturated aqueous sodium bicarbonate (18.75 L) was charged. The reaction mixture was stirred for 15 min, then stirring stopped and the layers allowed to separate. The lower aqueous layer was removed to waste. Saturated aqueous sodium chloride (18.75 L) was charged. The reaction mixture was stirred for 15 min, then stirring stopped and the layers allowed to separate. The lower aqueous layer was removed to waste. The crude product solution was dried by azeotropic distillation under reduced pressure to remove 7.5 L of toluene, giving 28.3 kg of a 14.08% w/w toluene solution of cycloheptanecarboxylic acid methyl ester.

### (b) 1-Phenyl-cycloheptanecarboxylic Acid Methyl Ester

Diisopropylamine (3.44 kg) and toluene (16.52 kg) were charged to a first reaction vessel and cooled to 0° C. with stirring. N-Hexyllithium (8.81 kg, 33% w/w) was added, maintaining a temperature of 5° C.±5° C. The mixture was stirred for 20 min at this temperature. Cycloheptanecarboxylic acid methyl ester (14.08% w/w in toluene; 26.93 kg) was first concentrated by removal of 11.37 L of toluene by distillation under reduced pressure, then charged to the first reaction vessel, maintaining a temperature of 5° C.±5° C.

The contents were allowed to warm to 20° C., stirred for 20 min at this temperature, then cooled back to 0° C. To a second reaction vessel was charged dibromo bis(tri-tert-butylphosphine) dipalladium (I) (Johnson Matthey Pd-113; 189.15 g); bromobenzene (3.06 L) and toluene (7.58 L) under an inert atmosphere at ambient temperature. The contents of the second vessel were charged to first vessel at a rate such that a temperature of 5° C.±5° C., was maintained, followed by line wash of toluene (3.79 L). The mixture was stirred at 0° C. for 1 hour, then allowed to warm to 20° C. and stirred at this temperature overnight. 2M hydrochloric acid (18.96 L) was added, whilst maintaining the temperature below 30° C., then the mixture was stirred at 20° C. for 15 min, stirring stopped and the layers allowed to separate. The lower aqueous layer was removed to waste. A second charge of 2M hydrochloric acid (18.96 L) was added, then the mixture stirred at 20° C. for 15 min, stirring stopped and the layers allowed to separate. The lower aqueous layer was removed to waste. Water (18.96 L) was charged, the mixture stirred at 20° C. for 15 min, then stirring stopped and the layers allowed to separate. The lower aqueous layer was removed to waste. The crude product solution was passed through cartridges containing Phosphonics SPM32 scavenger, then evaporated to dryness under reduced pressure on a rotating film evaporator to give 1-Phenyl-cycloheptanecarboxylic Acid methyl ester as a mobile brown oil (3.12 kg)

### (c) 1-Phenyl-cycloheptanecarboxylic Acid

Sodium Hydroxide (12.64 kg) was dissolved in water (31.60 L) and cooled to 20° C. 1-Phenyl-cycloheptanecarboxylic acid methyl ester (6.32 kg) in methanol (31.60 L) was added followed by a line rinse of methanol (5 L). The mixture was stirred at 60° C. for 18 hours, then cooled to 20° C. Concentrated hydrochloric acid (29.43 L) was added to precipitate the product, maintaining the temperature at below 50° C., then the mixture cooled to 20° C. and stirred for 18 hours. The crude product was collected by filtration and washed with water (31.60 L), then dispersed in methanol (37.41 L) and water (9.35 L). The mixture was heated with stirring to 62° C. at a rate of 1° C./min then cooled to 5° C. at a rate of 0.3° C./min and held at 5° C. overnight. The product was collected by filtration, washed with water (2×12.64 L) and dried in a vacuum oven at 40° C. for 72 hours to give 1-Phenyl-cycloheptanecarboxylic acid (5.60 kg).

### (d) 1-Phenyl-cycloheptanecarboxylic Acid (R)-(1-aza-bicyclo[2.2.2]oct-3-yl)ester

1-Phenyl-cycloheptanecarboxylic acid (2.70 kg) and butanenitrile (21.60 L). were charged to a first reaction vessel. The contents were heated to 70±5° C. with stirring to give a homogeneous solution. To a second reaction vessel was charged 1,1′-carbonyldiimidazole (1.1 equiv (molar); 2.16 kg) and butanenitrile (10.80 L). The contents were heated to 50±5° C. with stirring. The contents of the first vessel were transferred to the second vessel, the temperature raised to 70±5° C. and stirring continued for 30±15 min. (R)-(−)-3-quinuclidinol (1.67 kg) and butanenitrile (8.10 L), were charged to the first reaction vesel followed by potassium-t-amylate (7.32 L). This mixture was stirred for 15 min, then added to the second vessel, followed by a line rinse of butanenitrile (1.35 L). The mixture was stirred at 70° C. for 18 hours then cooled to 20° C. 1 M Hydrochloric acid (29.70 L) was charged, followed by sufficient concentrated hydrochloric acid to reduce the pH to below 7 (2.276 kg added). The mixture was stirred for 15 min, stirring stopped and the layers allowed to separate. The lower layer was removed to waste. Saturated aqueous sodium bicarbonate solution (27.00 L) was charged. The mixture was stirred for 15 min, stirring stopped and the layers allowed to separate. The lower layer was removed to waste. Solvent was removed by distillation under reduced pressure to give a 31.9% w/w solution of the sub-title product (10.73 kg of solution and 3.42 kg of product).

### (e) (R)-3-(1-Phenyl-cycloheptanecarbonyloxy)-1-(pyridin-2-ylcarbamoylmethyl)-1-azonia-bicyclo[2.2.2]octane bromide

To bromoacetic acid (3.00 kg) in methyl acetate (24.18 L) in a first reaction vessel was charged 1-propanephosphonic acid cyclic anhydride (T3P) (19.51 L) in methyl acetate (50% w/w solution). The contents were cooled to 5° C. with stirring, then a solution of 2-pyridinamine (8.13 kg) in methyl acetate (24.29 L) precooled to 10° C., was charged, keeping the temperature of the vessel contents below 5° C. The mixture was stirred for 1 hr, then stirring stopped and the layers allowed to separate. The lower layer was separated to waste. The remaining solution containing 2-bromo-N-pyridin-2-yl-acetamide (42.04 kg at 3.90% w/w) was transferred to a second reaction vessel and cooled to 0° C.

1-Phenyl-cycloheptanecarboxylic acid (R)-(1-aza-bicyclo[2.2.2]oct-3-yl)ester (31.9% w/w solution in butanenitrile; 7.88 kg) was charged to the second reaction vessel and the mixture stirred at 0° C. for 18 hours. The solid product was collected by filtration, washed with methyl acetate (6.22 L) and dried in a vacuum oven at 50° C. to give crude product (3.51 kg at 90.5% w/w 3.17 Kg overall).

Crude Product (3.48 kg) in ethanol (69.62 L) was heated to 75° C. until fully dissolved. The solution was filtered through a 1.2 micron filter, then cooled at a rate of 0.3° C./min to 0° C. and stirred at this temperature for 18 hours. The solid product was collected by filtration, washed with ethanol (7.47 L) and dried in a vacuum oven at 50° C. for 48 hours to give purified product (2.82 kg).

### Biological Activity of Muscarinic Antagonists

The inhibitory effects of compounds of the muscarinic antagonists were determined by a Muscarinic Receptor Radioligand Binding Assay. Radioligand binding studies utilising [³H]-N-methyl scopolamine ([³H]-NMS) and commercially available cell membranes expressing the human muscarinic receptors (M2 or M3) were used to assess the affinity of muscarinic antagonists for M2 and M3 receptors. Membranes in TRIS buffer were incubated in 96-well plates with [³H]-NMS and M3 antagonist at various concentrations for 3 hours. Membranes and bound radioligand were then harvested by filtration and allowed to dry overnight. Scintillation fluid was then added and the bound radioligand counted using a Can berra Packard Topcount scintillation counter

The half-life of antagonists at each muscarinic receptor was measured using the alternative radioligand [³H]-QNB and an adaptation of the above affinity assay. Antagonists were incubated for 3 hours at a concentration 10-fold higher than their Ki, as determined with the [³H]-QNB ligand, with membranes expressing the human muscarinic receptors. At the end of this time, [³H]-QNB was added to a concentration 25-fold higher than its Kd for the receptor being studied and the incubation continued for various time periods from 15 minutes up to 180 minutes. Membranes and bound radioligand were then harvested by filtration and allowed to dry overnight. Scintillation fluid was then added and the bound radioligand counted using a Can berra Packard Topcount scintillation counter.

The rate at which [³H]-QNB is detected binding to the muscarinic receptors is related to the rate at which the antagonist dissociates from the receptor, i.e. to the half life of the antagonists on the receptors.

Table 1 shows the IC₅₀ figures for Example 1.

TABLE 1

| Compound of | M3 |
|---|---|
| Example No. | pIC₅₀ |
| 1 | 10.1 |

Table 2 gives IC₅₀ strengths for the compounds of the examples.

TABLE 2

| Compound of | M3 |
|---|---|
| Example No. | pIC₅₀ |
| 3 | +++ |
| 8 | +++ |
| 9 | +++ |
| 10 | +++ |
| 11 | +++ |
| 12 | +++ |
| 13 | +++ |

M3 Binding IC₅₀ < 2 nM “+++”;
IC₅₀ 2-10 nM “++”;
IC₅₀ > 10 nM “+”;
NT—Not Tested.

### Preparation of β₂-Adrenoceptor Agonists

The following β₂-adrenoceptor agonists that may be employed in the combination of the present invention may be prepared as follows.

### General Experimental Details for Preparation of β₂-adrenoceptor for Agonists

¹H NMR spectra were recorded on a Varian Inova 400 MHz or a Varian Mercury-VX 300 MHz instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Column chromatography was carried out using silica gel (0.040-0.063 mm, Merck). Unless stated otherwise, starting materials were commercially available. All solvents and commercial reagents were of laboratory grade and were used as received.

The following method was used for LC/MS analysis:

Instrument Agilent 1100; Column Waters Symmetry 2.1×30 mm; Mass APCl; Flow rate
0.7 ml/min; Wavelength 254 nm; Solvent A: water+0.1% TFA; Solvent B: acetonitrile+0.1% TFA; Gradient 15-95%/B 8 min, 95% B 1 min.

Analytical chromatography was run on a Symmetry C₁₈-column, 2.1×30 mm with 3.5 μm particle size, with acetonitrile/water/0.1% trifluoroacetic acid as mobile phase in a gradient from 5% to 95% acetonitrile over 8 minutes at a flow of 0.7 ml/min.

The abbreviations or terms used in the examples have the following meanings:

SCX: Solid phase extraction with a sulfonic acid sorbent
HPLC: High performance liquid chromatography

### DMF: N,N-Dimethylformamide

The β₂-adrenoceptor agonists and the intermediates used in their preparation are herein named, based upon the structures depicted, using the IUPAC NAME, ACD Labs Version 8 naming package.

### β₂-Adrenoceptor Agonist 1: Preparation 1

### N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide

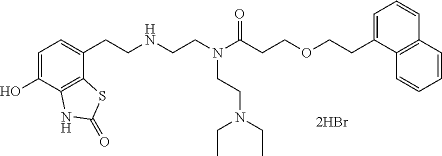

### a) tert-Butyl 3-[2-(1-naphthyl)ethoxy]propanoate

1-Naphthalene ethanol (10 g) was treated with benzyltrimethylammonium hydroxide (Triton B®; 0.9 mL of a 40% solution in methanol) and the resulting mixture stirred in vacuo for 30 minutes. The mixture was then cooled to 0° C. and treated with tert-butyl acrylate (8.19 g). The resulting mixture was slowly warmed to room temperature and stirred overnight. The crude mixture was subsequently absorbed onto aluminum oxide (30 g) and eluted with diethylether (200 mL). The organics were concentrated to give a crude material (16.6 g) which was purified by flash silica chromatography eluting with 1:8, diethylether: hexane to give the subtitled compound (12.83 g).

¹H NMR (CDCl₃) δ 8.05 (dd, 1H), 7.84 (dd, 1H), 7.72 (dd, 1H), 7.54-7.34 (m, 4H), 3.81-3.69 (m, 4H), 3.35 (t, 2H), 2.52-2.47 (m, 2H), 1.45 (s, 9H).

### b) 3-[2-(1-Naphthyl)ethoxy]propanoic acid

tert-Butyl 3-[2-(1-naphthyl)ethoxy]propanoate (6.19 g) was taken up in dichloromethane (30 mL) and treated with trifluoroacetic acid (5 mL). The resulting solution was stirred at room temperature for 2 hours, an additional 1 mL of trifluoroacetic acid was added and the solution stirred overnight. The mixture was concentrated, taken up in 2M sodium hydroxide solution (30 mL) and washed with ether (2×20 mL). The aqueous layer was subsequently acidified (using 1M hydrochloric acid) and extracted with ether (2×30 mL). The combined organics were washed with brine (20 mL), dried over anhydrous magnesium sulphate, filtered and concentrated in vacuo to give the sub-titled compound (5.66 g) as a clear oil.

¹H NMR (CDCl₃) δ 8.05 (bs, 1H), 7.85 (bs, 1H), 7.74 (bs, 1H), 7.50-7.38 (m, 4H), 3.84-3.75 (bm, 4H), 3.39 (bs, 2H), 2.65 (bs, 2H).

### c) N-(2-Diethylaminoethyl)-N-(2-hydroxyethyl)-3-[2-(1-naphthyl)ethoxy]-propanamide

Oxalyl chloride (0.33 g) was added dropwise to a solution of 3-[2-(1-naphthyl)ethoxy]propanoic acid (0.53 g) in dichloromethane (10 mL), dimethylformamide (1 drop) was added and stirring continued at room temperature for 1 hour. The mixture was subsequently concentrated, re-dissolved in dichloromethane (10 mL) and added dropwise to a solution of 2-(2-diethylaminoethylamino)ethanol (0.35 g) and diisopropylethylamine (0.56 g) in dichloromethane (10 mL). The resulting mixture was stirred at room temperature for 1 hour, diluted (dichloromethane, 50 mL), washed with water (2×20 mL), brine (20 mL), dried over magnesium sulfate and concentrated to give the crude product (0.91 g) which was purified by flash column chromatography (eluting with 5-7% methanol in dichloromethane) to give 0.63 g of the sub-titled compound.

¹H NMR (CDCl₃) δ 8.05 (d, 1H), 7.85 (d, 1H), 7.73 (d, 1H), 7.52-7.47 (m, 2H), 7.42-7.35 (m, 2H), 3.84-3.78 (m, 6H), 3.72-3.70 (m, 1/2H), 3.45-3.35 (m, 6H), 2.79-2.77 (m, 1+1/2H), 2.62-2.58 (m, 2H), 2.54-2.49 (m, 4H), 1.04-1.01 (m, 6H).

### d) N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide

A solution of dimethylsulfoxide (0.097 g) in dichloromethane (1 mL) was added to a solution of oxalyl chloride (0.079 g) in dichloromethane (10 mL) at −78° C. The reaction was stirred for 15 minutes and then a solution of N-(2-diethylaminoethyl)-N-(2-hydroxyethyl)-3-[2-(1-naphthyl)ethoxy]propanamide (0.22 g) in dichloromethane (1 mL+1 mL wash) was added and the reaction mixture stirred for a further 15 minutes. Triethylamine (0.29 g) was added and the reaction allowed to warm to room temperature over 1 hour, the mixture was subsequently diluted (dichloromethane 30 mL), the organics washed with sodium bicarbonate (20 mL), brine (20 mL), dried over anhydrous magnesium sulphate, filtered and concentrated in vacuo to give the sub-titled compound (0.21 g).

The crude product was dissolved in methanol (10 mL) and 7-(2-aminoethyl)-4-hydroxy-1,3-benthiazol-2(3H)-one hydrochloride (prepared according to the procedure outlined in Organic Process Research & Development 2004, 8(4), 628-642; 0.131 g) was added along with acetic acid (0.1 mL) and water (0.1 mL). After stirring at room temperature for 30 minutes, sodium cyanoborohydride (0.020 g) was added and the reaction mixture was stirred overnight. Ammonia (7N in methanol, 1 mL) was added and the mixture was concentrated. The crude residue was purified by flash column chromatography eluting with 1% ammonia; 5%-7% methanol in dichloromethane. The crude product was used directly in the next step.

### e) N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide

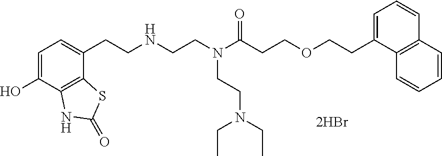

N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide (0.052 g) was dissolved in ethanol (1.5 mL) and treated with 48% hydrobromic acid (21 μl). The white solid dihydrobromide salt (0.058 g) was collected by filtration.

MS: APCI(+ve) 579 (M+1)

¹H NMR δ (DMSO) 11.78-11.71 (m, 1H), 10.11-10.06 (m, 1H), 9.51-9.43 (m, 0.33H), 9.21-9.13 (m, 0.66H), 8.75-8.66 (m, 1H), 8.59-8.51 (m, 1H), 8.06 (d, 1H), 7.95-7.90 (m, 1H), 7.79 (d, 1H), 7.60-7.48 (m, 2H), 7.47-7.39 (m, 2H), 6.87 (t, 1H), 6.76 (dd, 1H), 3.78-3.53 (m, 10H), 3.25-3.09 (m, 10H), 2.91-2.80 (m, 2H), 2.73-2.61 (m, 2H), 1.26-1.15 (m, 6H). NMR indicates approximately 2:1 mixture of rotamers at 298K.

### β₂-Adrenoceptor Agonist 1: (BA1): Preparation 2

### N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide

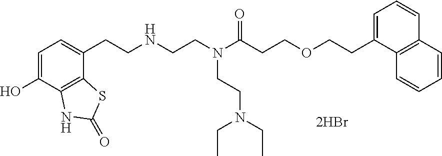

### a) N′-(2,2-Dimethoxyethyl)-N,N-diethyl-ethane-1,2-diamine

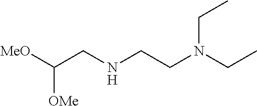

A solution of N,N-diethyl-ethylenediamine (150 g) in methanol (500 mL) was treated dropwise rapidly with glyoxal dimethylacetal (60 wt % soln. in water, 225 g) at 10-15° C. After the addition was complete the solution was warmed to 15° C., then to 22° C. and left at this temperature for 16 hours. The reaction mixture was treated with 5% palladium on carbon (Johnson-Matthey type 38H paste, 15 g) and hydrogenated at 6 bar until the reaction was complete as judged by GC/MS. The catalyst was removed by filtration and the filtrate evaporated to dryness (toluene azeotrope, 2.5 L), affording 196.2 g of the sub-titled compound.

¹H NMR (CDCl₃): 4.48 (t, 1H), 3.39 (s, 6H), 2.75 (d, 2H), 2.69 (t, 2H), 2.57-2.48 (m, 6H), 1.01 (ts, 6H).

### b) N-[2-(Diethylamino)ethyl]-N-(2,2-dimethoxyethyl)-3-[2-(1-naphthyl)ethoxy]propanamide

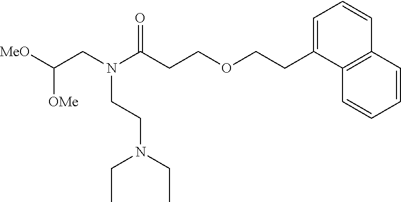

Oxalyl chloride (151 mL) was added dropwise over 45 minutes to a solution of 3-[2-(1-naphthyl)ethoxy]propanoic acid (389 g) (Example 7 step b)) in dichloromethane (2.1 L) and DMF (0.5 mL). The reaction mixture was stirred for a further 16 hours. The mixture was subsequently concentrated, redissolved in DCM (1.7 L) and added dropwise over 1.75 hours at 0° C. to a solution of N-(2,2-dimethoxyethyl)-N,N-diethylethane-1,2-diamine (325 g) and isopropyldiethylamine (551 mL) in DCM (1.7 L). The resulting mixture was stirred at room temperature for 3 hours, washed with aqueous saturated sodium bicarbonate solution (5×1 L), water (1.5 L) and dried over sodium sulphate and concentrated to give 650 g of the sub-titled compound.

m/e 431 (M+H⁺, 100%)

### c) N-[2-(Diethylamino)ethyl]-3-[2-(1-naphthyl)ethoxy]-N-(2-oxoethyl)propanamide

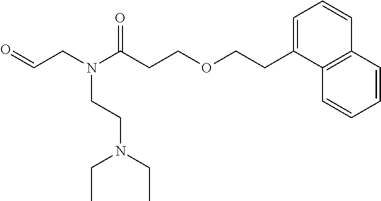

A solution of N-[2-(diethylamino)ethyl]-N-(2,2-dimethoxyethyl)-3-[2-(1-naphthyl)ethoxy]propanamide (93 g) in DCM (270 mL) was treated dropwise at 0° C. with trifluoroacetic acid (270 mL) over 1.5 hours. After the addition the reaction mixture was allowed to warm to room temperature and stirred for a further 1 hour. The reaction mixture was concentrated and the residue poured into aqueous saturated sodium bicarbonate solution (1800 mL, caution). The aqueous mixture was extracted with DCM (4×400 mL) and the combined extracts were dried over magnesium sulphate and concentrated. The residue was used directly in the following reaction.

### d) N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide

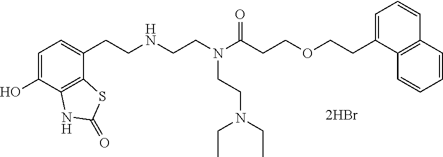

A suspension of 7-(2-amino-ethyl)-4-hydroxy-3H-benzothiazol-2-one hydrochloride (53 g) in dry NMP (216 mL) was heated to 60° C. and treated in one portion with a solution of NaOH (8.2 g) in methanol (102 mL). The bright orange suspension was cooled to room temperature and treated dropwise with a solution of N-[2-(diethylamino)ethyl]-3-[2-(1-naphthyl)ethoxy]-N-(2-oxoethyl)propanamide in dichloromethane (475 mL) over 20 minutes. The reaction was left to stir for 25 minutes. Sodium triacetoxyborohydride (91.5 g) was then added in portions over 20 minutes and the mixture stirred for a further 50 minutes. The reaction mixture was poured into water (1.8 L) and the acidic solution (pH5) was washed with tert. butyl methyl ether (TBME) (3×500 mL). The aqueous phase was basified to pH8 by the addition of solid potassium carbonate and extracted with dichloromethane (3×750 mL); the combined organic extracts were dried over magnesium sulphate and concentrated to give a dark oil. This was dissolved in ethanol (200 mL) and 48% aqueous hydrobromic acid (73 mL) was added. The solution was aged for 30 minutes then evaporated to dryness. The residue was triturated with ethanol (560 mL); the resultant solid was collected by filtration and dried in vacuo at 50° C. The sticky solid was suspended in boiling ethanol (100 mL) and filtered while hot. The collected solid was dried in vacuo at 50° C. This material was recrystallised from ethanol/water (3:1, 500 mL). After standing overnight the resultant solid was collected by filtration and washed with ice-cold ethanol (75 mL). Drying in vacuo at 50° C. for 24 hr afforded 57 g of the title compound.

### β₂-Adrenoceptor Agonist 2: (BA2)

### N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide dihydrobromide

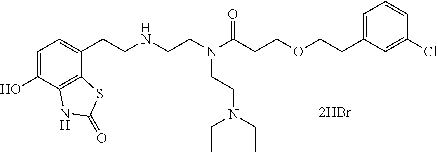

### a) tert-Butyl 3-[2-(3-chlorophenyl)ethoxy]propanoate

2-(3-chlorophenyl)ethanol (20 g) was treated with benzyltrimethylammonium hydroxide (Triton B®) (2.67 mL) and the resultant mixture was stirred in vacuo for 30 minutes. The mixture was then cooled to 0° C. and treated with t-butyl acrylate (17.40 g). The reaction was warmed to room temperature and stirred for 16 hours. The mixture was filtered through aluminum oxide (15 g) eluting with ether (75 mL). The collected filtrate was concentrated to give the sub-titled compound (34.40 g) as an oil.

¹H NMR (CDCl₃) [symbol] 7.26-7.07 (m, 4H), 3.69-3.59 (m, 4H), 2.86-2.81 (t, 2H), 2.50-2.45 (t, 2H), 1.43 (s, 9H)

### b) 3-[2-(3-chlorophenyl)ethoxy]propanoic acid

tert-Butyl 3-[2-(3-chlorophenyl)ethoxy]propanoate (example 1a), 34.40 g) was dissolved in dichloromethane (150 mL) and treated with trifluoroacetic acid (50 mL). The mixture was stirred at room temperature for 3 hours, then concentrated in vacuo and azeotroped with dichloromethane (2×10 mL). The residue was taken up in dichlormethane (300 mL) and extracted with saturated sodium hydrogen carbonate (200 mL). The basic layer was washed with dichloromethane (20 mL) then acidified with 2M hydrochloric acid. The acidic layer was extracted with dichloromethane (2×200 mL). The organic layers were combined, washed with brine, dried over anhydrous magnesium sulphate, filtered and concentrated to yield the sub-titled compound (24.50 g) as an oil.

m/e 227 [M−H]

### c) N-[2-(Diethylamino)ethyl]-N-(2,2-dimethoxyethyl)-3-[2-(3-chlorophenyl)ethoxy]propanamide

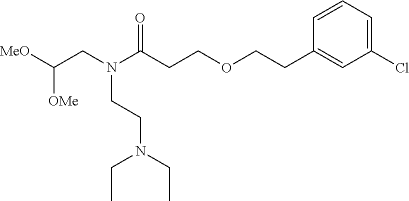

Oxalyl chloride (9.50 mL) was added dropwise over 45 minutes to a solution of 3-[2-(3-chlrophenyl)ethoxy]propanoic acid (22.50 g) (example 1b) in dichloromethane (120 ml) and DMF (0.5 mL). The reaction mixture was stirred for a further 16 hours. The mixture was subsequently concentrated, redissolved in DCM (1.7 L) and added dropwise over 1.75 hours at 0° C. to a solution of N-(2,2-dimethoxyethyl)-N,N-diethylethane-1,2-diamine (20.20 g)(example 16a) and isopropyldiethylamine (34.43 mL) in DCM (200 mL). The resulting mixture was stirred at room temperature for 16 hours, washed with aqueous saturated sodium bicarbonate solution (3×1 L), water (1.5 L) and dried over sodium sulphate and concentrated to give 39.50 g of the sub-titled compound.

m/e 415 (M+H⁺, 83%)

### d) N-[2-(Diethylamino)ethyl]-3-[2-(3-chlorophenyl)ethoxy]-N-(2-oxoethyl)propanamide

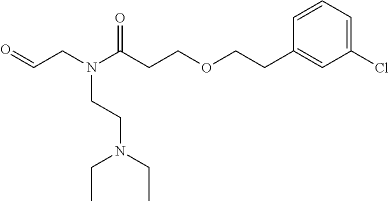

A solution of N-[2-(Diethylamino)ethyl]-N-(2,2-dimethoxyethyl)-3-[2-(3-chlorophenyl)ethoxy]propanamide (example 1c) (20 g) in DCM (500 mL) was treated dropwise at 0° C. with trifluoroacetic acid (50 mL) over 30 minutes. After the addition the reaction mixture was allowed to warm to room temperature and stirred for a further 1 hour. The reaction mixture was concentrated and the residue poured into aqueous saturated sodium bicarbonate solution (1800 mL, caution). The aqueous mixture was extracted with DCM (3×400 mL) and the combined extracts were dried over magnesium sulphate and concentrated. The residue was used directly in the following reaction.

### e) N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide dihydrobromide

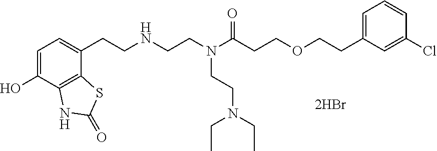

A suspension of 7-(2-amino-ethyl)-4-hydroxy-3H-benzothiazol-2-one hydrochloride (11.77 g) in dry NMP (50 mL) was heated to 65° C. and treated in one portion with a solution of NaOH (1.83 g) in methanol (23 mL). The bright orange suspension was cooled to room temperature and treated dropwise with a solution of N-[2-(diethylamino)ethyl]-3-[2-(3-chlorophenyl)ethoxy]-N-(2-oxoethyl)propanamide (example 1d) in dichloromethane (50 mL) over 30 minutes. The reaction was left to stir for 30 minutes. Sodium triacetoxyborohydride (20.33 g) was then added in portions over 20 minutes and the mixture stirred for a further 16 hours. The reaction mixture was poured into water (1.8 L), basified to pH8 by the addition of solid potassium carbonate and extracted with dichloromethane (2×500 mL); the combined organic extracts were dried over magnesium sulphate and concentrated to give a dark oil. The residue was purified by chromatography on silica with 10% (0.1% aq NH₃/MeOH)/DCM as eluent to give the sub-title compound as a brown oil. Yield (6.58 g). This was dissolved in ethanol (150 mL) and 48% aqueous hydrobromic acid (10 mL) was added. The solution was aged for 30 minutes then evaporated to dryness. The residue was triturated with ethanol (100 mL); the resultant solid was collected by filtration and dried in vacuo at 50. This material was recrystallised from ethanol/water (6:1, 500 mL); after standing overnight the resultant solid was collected by filtration and washed with ice-cold ethanol (75 mL). Drying in vacuo at 50° C. for 24 hr afforded 4.96 g of the title compound.

MS: APCI (+ve): 563 (M+1) 99.3% purity (T9505M).

¹H NMR (DMSO, 90° C.), δ 11.75-11.73 (m, 1H), 10.08-10.06 (d, 1H), 8.65 (bs, 1H), 7.33-7.19 (m, 4H), 6.89-6.84 (t, 1H), 6.77-6.74 (m, 1H), 3.68-3.58 (m, 8H), 3.17-3.16 (m, 10H), 2.86-2.80 (m, 4H), 2.67-2.62 (m, 2H), 1.23-1.19 (t, 6H).

Elemental Analysis

CHNS C: 46.54% (46.39); H: 5.75% (5.70); N, 7.94% (7.73); S: 4.46% (4.42)

### β₂-Adrenoceptor Agonist 3: (BA3)

### 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one dihydrobromide

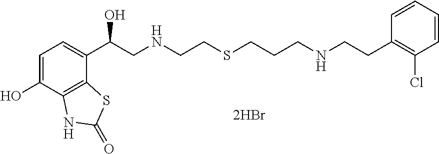

### a) 1-Chloro-2-[(E)-2-nitrovinyl]benzene

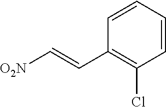

2-Chlorobenzaldehyde (ex Aldrich) (10.0 g) was mixed with nitromethane (26.05 g) and ammonium acetate (21.92 g) in acetic acid (200 mL), and the mixture was heated at reflux for 40 minutes. The mixture was allowed to cool to room temperature, and the majority of the acetic acid was removed in vacuo. The residue was dissolved in dichloromethane and washed with water, then potassium carbonate solution (×2), then water again. The organics were dried over anhydrous magnesium sulfate, filtered and evaporated to give the desired material, as an orange oil (12.83 g).

¹H NMR δ (CDCl₃) 8.41 (d, 1H), 7.62-7.57 (m, 2H), 7.52-7.48 (m, 1H), 7.43 (dt, 1H), 7.34 (ddd, 1H)

### b) 2-(2-Chlorophenyl)ethanamine

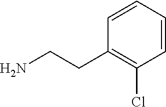

Aluminum hydride was prepared by the drop-wise addition of a solution of sulphuric acid (8.40 mL) in dry THF (60 mL) to a stirred solution of 1.0M lithium aluminum hydride in THF (314 mL), at 0-10° C., under a nitrogen atmosphere. After stirring at 5° C. for 30 minutes, a solution of 1-chloro-2-[(E)-2-nitrovinyl]benzene (12.83 g) in dry THF (160 mL) was added dropwise maintaining the internal temperature between 0° C. and 10° C. When the addition was complete the reaction was heated at reflux for 5 minutes. The mixture was allowed to cool to room temperature, then cooled to 0° C. and isopropanol (22 mL) carefully added dropwise maintaining the temperature below 20° C. 2M Sodium hydroxide (35 mL) was carefully added dropwise maintaining the temperature below 20° C. The mixture was stirred at room temperature for 30 minutes, then filtered through a layer of celite, which was then washed with THF (×3). The filtrate was evaporated to dryness. The residue was purified using silica column chromatography, using ethyl acetate to load the material, then 10% triethylamine in ethyl acetate, followed by 10% triethylamine in 45% ethanol: 45% ethyl acetate as the eluents, to give the desired material (4.66 g).

¹H NMR δ (CDCl₃) 7.36 (dd, 1H), 7.25-7.13 (m, 3H), 2.98 (dt, 2H), 2.91-2.87 (m, 2H)

### c) tert-Butyl [2-(2-chlorophenyl)ethyl]carbamate

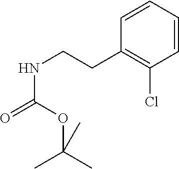

To a stirred solution of 2-(2-chlorophenyl)ethanamine (25.57 g) and triethylamine (22.87 mL) in dry THF (300 mL) was added a solution of di-tert-butyl dicarbonate (35.85 g) in dry THF (50 mL) over 10 minutes, at ambient temperature, under a nitrogen atmosphere. The reaction mixture was stirred at room temperature for 3 hours. The solvents were removed in vacuo to give the desired material, as a yellow oil (42.0 g).

¹H NMR δ (CDCL3) 7.35 (d, 1H), 7.25-7.14 (m, 3H), 4.57 (s, 1H), 3.43-3.35 (m, 2H), 2.95 (t, 2H), 1.43 (d, 9H)

### d) tert-Butyl allyl[2-(2-chlorophenyl)ethyl]carbamate

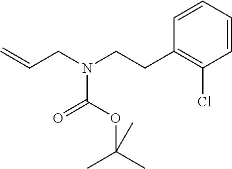

To a suspension of sodium hydride (60% in mineral oil) (7.23 g), which had been washed with ether (×3), in dry DMF (200 mL) was added a solution of tert-butyl [2-(2-chlorophenyl)ethyl]carbamate (42.0 g) in dry DMF (50 mL), over a 15 minute period, at 35° C., under a nitrogen atmosphere. When the addition was complete, the mixture was stirred at 50° C. for 90 minutes. The mixture was allowed to cool to room temperature, then allyl bromide (15.63 mL) was added slowly, keeping the temperature at 25° C., using external cooling. The mixture was stirred at room temperature for 2 hours, then diluted with water and extracted with ethyl acetate (×3). The organics were combined, washed with water, dried over anhydrous magnesium sulfate, filtered and evaporated. The residue was purified using silica column chromatography, loading with 1% ethyl acetate in isohexane, then using isohexane with ethyl acetate (0%, 1%, 2%, %5) as the eluents to give the desired material (27.0 g). There were several mixed fractions, so these were combined, and re-purified using silica column chromatography, as above, to give a further 4 g of desired material. Both crops of product were combined to give 31.0 g in total.

¹H NMR δ (CDCl₃) 7.36-7.31 (m, 1H), 7.21-7.12 (m, 3H), 5.83-5.68 (m, 1H), 5.17-5.05 (m, 2H), 3.86-3.66 (m, 2H), 3.41 (t, 2H), 3.03-2.90 (m, 2H), 1.43 (s, 9H)

¹H HPLC: 95.90% @ 220 nm [M+H-Boc]+=196.1 (Calc=295.1339) (multimode+)

### e) tert-Butyl[2-(2-chlorophenyl)ethyl]{3-[(2-hydroxyethyl)thio]propyl}carbamate

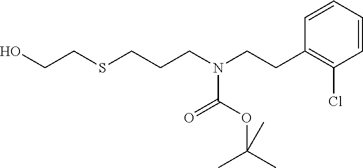

tert-Butyl allyl[2-(2-chlorophenyl)ethyl]carbamate (31.0 g) was mixed with 2-mercaptoethanol (7.37 mL), and AIBN (1.15 g), and stirred at 65° C. for 45 minutes. The mixture was cooled and more mercaptoethanol (1 mL) and AIBN (200 mg) added. The mixture was then heated at 65° C. for a further 30 minutes. The material was purified by silica column chromatography, loading the material in 20% ethyl acetate in isohexane, then eluting with 20% ethyl acetate in isohexane, changing to 50%, to give the desired material (31.94 g).

¹H NMR δ (CDCl₃) 7.38-7.32 (m, 1H), 7.22-7.13 (m, 3H), 3.75-3.68 (m, 2H), 3.41 (t, 2H), 3.32-3.14 (m, 2H), 3.03-2.91 (m, 2H), 2.72 (t, 2H), 2.54-2.36 (m, 2H), 1.85-1.71 (m, 2H), 1.42 (s, 9H)

¹H HPLC: 92.31% @ 220 nm [M+H-Boc]+=274.1 (Calc=373.1478) (multimode+)

### f) tert-Butyl[2-(2-chlorophenyl)ethyl]{3-[(2-oxoethyl)thio]propyl}carbamate

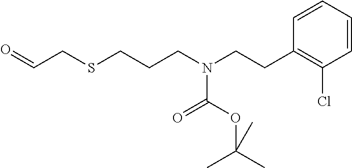

Sulfur trioxide:pyridine complex (30.52 g) was dissolved in DMSO (200 mL) and stirred at room temperature, under a nitrogen atmosphere, for 15 minutes. DCM (100 mL) was added, followed by a solution of tert-butyl[2-(2-chlorophenyl)ethyl]{3-[(2-hydroxyethyl)thio]propyl}carbamate (23.9 g) and Hunigs base (63.5 mL) in DCM (160 mL), which was added in one portion (exotherm). The resulting mixture was stirred at ambient temperature for 15 minutes. The reaction mixture was diluted with ethyl acetate, washed with water, then 1N HCl, then saturated sodium bicarbonate solution, dried over anhydrous magnesium sulfate, filtered and the solvents removed in vacuo. The material was purified by silica column chromatography eluting with 20% ethyl acetate in isohexane to give the desired material (12.43 g).

¹H NMR δ (CDCl₃) 9.46 (t, 1H), 7.36-7.32 (m, 1H), 7.21-7.13 (m, 3H), 3.40 (t, 2H), 3.29-3.13 (m, 4H), 3.02-2.90 (m, 2H), 2.45-2.34 (m, 2H), 1.82-1.69 (m, 2H), 1.49-1.36 (m, 9H)

### g) tert-Butyl[2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

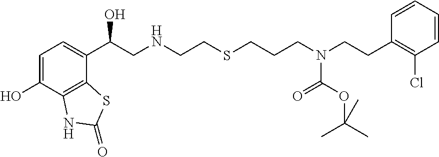

The tert-butyl[2-(2-chlorophenyl)ethyl]{3-[(2-oxoethyl)thio]propyl}carbamate (11.32 g) was dissolved in a mixture of methanol (200 mL) and acetic acid (1.74 ml). 7-[(1R)-2-amino-1-hydroxyethyl]-4-hydroxy-1,3-benzothiazol-2(3H)-one hydrochloride (8.0 g) was added to the solution, and the mixture stirred at room temperature, under a nitrogen atmosphere, for 1 hour. Sodium cyanoborohydride (1.92 g) was added and the mixture stirred for a further 2 hours. The solvents were removed in vacuo, and the residue diluted with water, basified with 0.880 aqueous ammonia, and extracted with ethyl acetate (×3) (filtered through celite during extraction). The organics were combined, washed with brine, dried over anhydrous sodium sulfate, filtered and evaporated to give a brown residue (15.5 g). The material was purified using silica column chromatography, using DCM with MeOH (2%, 5%, 10%, 20% and 30%, all with 1% 0.880 aq NH₃) as the eluent, to give the desired material (6.67 g) (38% yield)

¹H NMR δ (DMSO) 7.43-7.38 (m, 1H), 7.30-7.21 (m, 3H), 6.86 (d, 1H), 6.69 (d, 1H), 4.56 (dd, 1H), 3.23-3.10 (m, 2H), 2.88 (t, 2H), 2.71-2.48 (m, 8H), 2.46-2.39 (m, 2H), 1.72-1.62 (m, 2H), 1.40-1.22 (m, 9H)

¹H HPLC: 97.46% @ 220 nm [M+H]⁺=582.1 (Calc=582.1863) (multimode+)

### h) 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one dihydrobromide

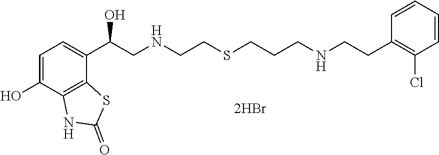

To a stirred suspension of the Boc compound from part g) (5.93 g) in DCM (20 mL) was added trifluoroacetic acid (20 mL) at 0° C., and the resulting mixture was stirred under nitrogen for 30 minutes. The mixture was diluted with toluene, and solvents removed, then azeotroped with toluene (×2). The residue was dissolved in acetonitrile, acidified with 48% aq HBr and concentrated in vacuo (not to dryness). The mixture was further diluted with acetonitrile and the precipitated solid collected by filtration, washed with acetonitrile and dried under vacuum to give 6.35 g. A 3.8% impurity was present (isomer from part e)), so the material was redissolved in a 1:1 mixture of acetonitrile:water and purified using prep HPLC (Sunfire 30×80 mm C8 column; NH₄OAc buffer; acetonitrile 5-50% over 10 minutes). The resultant material was dried overnight in a dessicator at 10 mbar over KOH and H₂SO₄. The resulting di-acetate salt was dissolved in water and basified with 0.880 aq ammonia. A white gum formed, so the aqueous was decanted off, and the gum dried in vacuo to give the free base (4.11 g). This was dissolved in hot ethanol, and the solution was filtered, then allowed to cool to room temperature. The solution was acidified with 48% aq. HBr and left to crystallize. The white solid was collected by filtration, washed with ethanol and dried in vacuo to give 3.81 g Crop 1.

¹H NMR δ (DMSO) 11.67 (s, 1H), 10.15 (s, 1H), 8.70 (s, 4H), 7.50-7.30 (m, 4H), 6.94 (d, 1H), 6.78 (d, 1H), 6.45 (s, 1H), 4.96-4.90 (m, 1H), 3.22-3.02 (m, 10H), 2.86-2.76 (m, 2H), 2.66 (t, 2H), 1.91 (quintet, 2H)

HPLC: 99.63% @ 220 nm [M+H]⁺=482 (calc=482.1339) (MultiMode+)

Elemental analysis:

| | | C | H | N | S |
|---|---|---|---|---|---|
| Calculated: | | 41.04 | 4.70 | 6.53 | 9.96 |
| Found: | 1: | 41.07 | 4.69 | 6.67 | 9.72 |
| | 2: | 41.08 | 4.68 | 6.74 | 9.67 |
| | 3: | 40.96 | 4.68 | 6.75 | 9.67 |

The mother liquors were evaporated to dryness then triturated with acetonitrile. The solid was collected by filtration to give 719 mg Crop 2 (4.53 g total).

¹H NMR δ (DMSO) 11.67 (s, 1H), 10.15 (s, 1H), 8.80-8.60 (m, 4H), 7.50-7.29 (m, 4H), 6.94 (d, 1H), 6.78 (d, 1H), 6.45 (s, 1H), 4.96-4.89 (m, 1H), 3.22-3.00 (m, 10H), 2.85-2.76 (m, 2H), 2.66 (t, 2H), 1.90 (quintet, 2H)

HPLC: 99.20% @ 220 nm [M+H]⁺=482 (calc=482.1339) (MultiMode+)

Elemental analysis:

| | | C | H | N | S |
|---|---|---|---|---|---|
| Calculated: | | 41.04 | 4.70 | 6.53 | 9.96 |
| Found: | 1: | 40.90 | 4.69 | 6.78 | 9.60 |
| | 2: | 41.01 | 4.70 | 6.83 | 9.60 |
| | 3: | 40.97 | 4.69 | 6.76 | 9.63 |

### Biological Activity of B₂-Adrenoceptor Agonists

Adrenergic 62 Mediated cAMP Production

### Cell Preparation

H292 cells were grown in 225 cm2 flasks incubator at 37° C., 5% CO₂ in RPMI medium containing, 10% (v/v) FBS (foetal bovine serum) and 2 mM L-glutamine.

### Experimental Method

Adherent H292 cells were removed from tissue culture flasks by treatment with Accutase™ cell detachment solution for 15 minutes. Flasks were incubated for 15 minutes in a humidified incubator at 37° C., 5% CO₂. Detached cells were re-suspended in RPMI media (containing 10% (v/v) FBS and 2 mM L-glutamine) at 0.05×10⁶ cells per mL. 5000 cells in 100 μL were added to each well of a tissue-culture-treated 96-well plate and the cells incubated overnight in a humidified incubator at 37° C., 5% CO₂. The culture media was removed and cells were washed twice with 100 μL assay buffer and replaced with 50 μL assay buffer (HBSS solution containing 10 mM HEPES pH7.4 and 5 mM glucose). Cells were rested at room temperature for 20 minutes after which time 25 μL of rolipram (1.2 mM made up in assay buffer containing 2.4% (v/v) dimethylsulphoxide) was added. Cells were incubated with rolipram for 10 minutes after which time Compound A was added and the cells were incubated for 60 minutes at room temperature. The final rolipram concentration in the assay was 300 μM and final vehicle concentration was 1.6% (v/v) dimethylsulphoxide. The reaction was stopped by removing supernatants, washing once with 100 μL assay buffer and replacing with 50 μL lysis buffer. The cell monolayer was frozen at −80° C. for 30 minutes (or overnight).

AlphaScreen™ cAMP Detection

The concentration of cAMP (cyclic adenosine monophosphate) in the cell lysate was determined using AlphaScreen™ methodology. The frozen cell plate was thawed for 20 minutes on a plate shaker then 10 μL of the cell lysate was transferred to a 96-well white plate. 40 μL of mixed AlphaScreen™ detection beads pre-incubated with biotinylated cAMP, was added to each well and the plate incubated at room temperature for 10 hours in the dark. The AlphaScreen™ signal was measured using an EnVision spectrophotometer (Perkin-Elmer Inc.) with the recommended manufacturer's settings. cAMP concentrations were determined by reference to a calibration curve determined in the same experiment using standard cAMP concentrations. A concentration response curve for Compound A was constructed and data was fitted to a four parameter logistic equation to determine both the pEC₃₀ and Intrinsic Activity. Intrinsic Activity was expressed as a fraction relative to the maximum activity determined for formoterol in each experiment. Result are in Table 1.

### Selectivity Assays

### Adrenergic α1D

### Membrane Preparation

Membranes were prepared from human embryonic kidney 293 (HEK293) cells expressing recombinant human α1_{D} receptor. These were diluted in Assay Buffer (50 mM HEPES, 1 mM EDTA, 0.1% gelatin, pH 7.4) to provide a final concentration of membranes that gave a clear window between maximum and minimum specific binding.

### Experimental Method

Assays were performed in U-bottomed 96-well polypropylene plates. 10 μIL [³H]-prazosin (0.3 nM final concentration) and 10 μL of Compound A (10× final concentration) were added to each test well. For each assay plate 8 replicates were obtained for [³H]-prazosin binding in the presence of 10 μL vehicle (10% (v/v) DMSO in Assay Buffer; defining maximum binding) or 10 μL BMY7378 (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-prazosin binding) were determined using serial dilutions typically in the range 0.1 nM to 10 μM. Data was fitted to a four parameter logistic equation to determine the compound potency, which was expressed as pIC50 (negative log molar concentration inducing 50% inhibition of [³H]-prazosin binding). Results are shown in Table 1 below.

### Adrenergic β1

### Membrane Preparation

Membranes containing recombinant human adrenergic beta 1 receptors were obtained from Euroscreen. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 10 μL [¹²⁵I]-Iodocyanopindolol (0.036 nM final concentration) and 10 μL of Compound A (10× final concentration) were added to each test well. For each assay plate 8 replicates were obtained for [¹²⁵I]-Iodocyanopindolol binding in the presence of 10 μL vehicle (10% (v/v) DMSO in Assay Buffer; defining maximum binding) or 10 μL Propranolol (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [¹²⁵I]-Iodocyanopindolol binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [¹²⁵I]-Iodocyanopindolol binding). Results are shown in Table 1 below.

### Dopamine D2

### Membrane Preparation

Membranes containing recombinant human Dopamine Subtype D2s receptors were obtained from Perkin Elmer. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 30 μL [³H]-spiperone (0.16 nM final concentration) and 30 μL of Compound A (10× final concentration) were added to each test well. For each assay plate 8 replicates were obtained for [³H]-spiperone binding in the presence of 30 μL vehicle (10% (v/v) DMSO in Assay Buffer; defining maximum binding) or 30 μL Haloperidol (10 μM final concentration; defining non-specific binding (NSB)). Membranes were then added to achieve a final volume of 300 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of Bₒ. Compound concentration-effect curves (inhibition of [³H]-spiperone binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [³H]-spiperone binding). Results are shown in Table 3.

TABLE 3

| | | | α1 bind | | D2 bind |
|---|---|---|---|---|---|
| Compound | β2 pEC50 | β2 Int Act | pIC50 | β1 bind p IC50 | pIC50 |
| BA1 | 8.2 | 0.8 | 6.6 | <5 | 6.1 |
| BA2 | 8.3 | 0.7 | <6.1 | <5 | 5.6 |
| BA3 | 9.2 | 0.8 | 7.6 | 6.9 | 5.8 |

### Combination Data

### Evaluation of Bronchodilator Activity in the Guinea Pig Isolated Tracheal Ring Preparation.

Guinea pigs (300-500 g) were killed by cervical dislocation and the trachea was isolated. The trachea was cut into segments 2-3 cartilage rings in width and suspended in 10 ml organ baths in modified Krebs' solution (mM; NaCl, 90; NaHCO₃, 45; KCl, 5; MgSO₄.7H₂O, 0.5; Na₂HPO₄.2H₂O, 1; CaCl₂, 2.25; glucose, 10; pH 7.4 gassed with 5% CO₂, 95% O₂ at 37° C.). The tracheal rings were attached to an isometric force transducer for the measurement of isometric tension. The tissues were washed and a force of 1 g was applied to each tissue. The rings were contracted with methacholine (1 μM). Once the contraction had reached a plateau, vehicle (0.01% DMSO in distilled H₂O), indacaterol (10 nM), N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide (10 nM), N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide di-D-mandelate salt (1 nM), (R)-3-(1-Phenyl-cycloheptanecarbonyloxy)-1-(pyridin-2-ylcarbamoylmethyl)-1-azonia-bicyclo[2.2.2]octane bromide (1 nM), a combination of (R)-3-(1-Phenyl-cycloheptanecarbonyloxy)-1-(pyridin-2-ylcarbamoylmethyl)-1-azonia-bicyclo[2.2.2]octane bromide (1 nM) and indacaterol (10 nM), a combination of N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide (10 nM) and (R)-3-(1-Phenyl-cycloheptanecarbonyloxy)-1-(pyridin-2-ylcarbamoylmethyl)-1-azonia-bicyclo[2.2.2]octane bromide (1 nM) and, or a combination of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt (1 nM) and (R)-3-(1-Phenyl-cycloheptanecarbonyloxy)-1-(pyridin-2-ylcarbamoylmethyl)-1-azonia-bicyclo[2.2.2]octane bromide (1 nM) was added and the tissue left for 60 min. The tension was measured in each ring at 60 min following compound addition and was expressed as a % relaxation of the constriction to methacholine (1 μM) (mean±s.e.mean). Data were collected using the Chart 4 software (ADInstruments, Charlgrove, UK).

Assessment of the combination of indacaterol and (R)-3-(1-Phenyl-cycloheptanecarbonyloxy)-1-(pyridin-2-ylcarbamoylmethyl)-1-azonia-bicyclo[2.2.2]octane bromide:

The relaxation (expressed as a percentage of the maximum response to methacholine (1 μM)) to indacaterol (10 nM) was 24±6.9, the percentage relaxation to (R)-3-(1-Phenyl-cycloheptanecarbonyloxy)-1-(pyridin-2-ylcarbamoylmethyl)-1-azonia-bicyclo[2.2.2]octane bromide: (1 nM) was 9±9.4 and the percentage relaxation to a combination of indacaterol (10 nM) and (R)-3-(1-Phenyl-cycloheptanecarbonyloxy)-1-(pyridin-2-ylcarbamoylmethyl)-1-azonia-bicyclo[2.2.2]octane bromide: (1 nM) was 40±3.6. The percentage relaxation to vehicle was 0±0 (n=3; see FIG. 9, wherein Compound Z is (R)-3-(1-Phenyl-cycloheptanecarbonyloxy)-1-(pyridin-2-ylcarbamoylmethyl)-1-azonia-bicyclo[2.2.2]octane bromide).

Assessment of the combination of N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide and (R)-3-(1-Phenyl-cycloheptanecarbonyloxy)-1-(pyridin-2-ylcarbamoylmethyl)-1-azonia-bicyclo[2.2.2]octane bromide:

The relaxation (expressed as a percentage of the maximum response to methacholine (1 μM)) to N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide (10 nM) was 18±11.2, the percentage relaxation to (R)-3-(1-Phenyl-cycloheptanecarbonyloxy)-1-(pyridin-2-ylcarbamoylmethyl)-1-azonia-bicyclo[2.2.2]octane bromide: (1 nM) was 9±4.3 and the percentage relaxation to a combination of N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide (10 nM) and (R)-3-(1-Phenyl-cycloheptanecarbonyloxy)-1-(pyridin-2-ylcarbamoylmethyl)-1-azonia-bicyclo[2.2.2]octane bromide: (1 nM) was 32±14.1. The percentage relaxation to vehicle was 6±4.5 (n=4; see FIG. 10 where compound V is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide dihydrobromide and compound Z is (R)-3-(1-Phenyl-cycloheptanecarbonyloxy)-1-(pyridin-2-ylcarbamoylmethyl)-1-azonia-bicyclo[2.2.2]octane bromide).

### Assessment of the combination of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide di-D-mandelate and (R)-3-(1-Phenyl-cycloheptanecarbonyloxy)-1-(pyridin-2-ylcarbamoylmethyl)-1-azonia-bicyclo[2.2.2]octane bromide:

The relaxation (expressed as a percentage of the maximum response to methacholine (1 μM)) to N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate (1 nM) was 23±10, the percentage relaxation to (R)-3-(1-Phenyl-cycloheptanecarbonyloxy)-1-(pyridin-2-ylcarbamoylmethyl)-1-azonia-bicyclo[2.2.2]octane bromide: (1 nM) was 5±1.8 and the percentage relaxation to a combination of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide di-D-mandelate (1 nM) and (R)-3-(1-Phenyl-cycloheptanecarbonyloxy)-1-(pyridin-2-ylcarbamoylmethyl)-1-azonia-bicyclo[2.2.2]octane bromide: (1 nM) was 42±11.1. The percentage relaxation to vehicle was 6±4.5 (n=4; see FIG. 11 where compound W is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide di-D-mandelate and compound Z is (R)-3-(1-Phenyl-cycloheptanecarbonyloxy)-1-(pyridin-2-ylcarbamoylmethyl)-1-azonia-bicyclo[2.2.2]octane bromide)

### In Vivo Combination Data

### Evaluation of Lung Function in Anaesthetised Guinea Pigs

Male Dunkin-Hartley guinea pigs (300-600 g) are weighed and dosed with vehicle (0.05M phosphate, 0.1% Tween 80, 0.6% saline, pH 6) or compound via the intratracheal route under recoverable gaseous anaesthesia (5% halothane in oxygen). Animals are dosed with compound or vehicle two hours prior to the administration of methacholine. Guinea pigs are anaesthetised with pentobarbitone (1 mL/kg of 60 mg/mL solution i.p.) approximately 30 minutes prior to the first bronchoconstrictor administration. The trachea is cannulated and the animal ventilated using a constant volume respiratory pump (Harvard Rodent Ventilator model 683) at a rate of 60 breath/min and a tidal volume of 5 mL/kg. A jugular vein is cannulated for the administration of methacholine or maintenance anaesthetic (0.1 mL of pentobarbitone solution, 60 mg/mL, as required).

The animals are transferred to a Flexivent System (SCIREQ, Montreal, Canada) in order to measure airway resistance. The animals are ventilated (quasi-sinusoidal ventilation pattern) at 60 breaths/min at a tidal volume of 5 mL/kg. A positive end expiratory pressure of 2-3 cm H₂O was applied. Respiratory resistance is measured using the Flexivent “snapshot” facility (1 second duration, 1 Hz frequency). Once a stable baseline resistance value has been obtained the animals are given methacholine in ascending doses (0.5, 1, 2, 3 and 5 μg/kg, i.v) at approximately 4-minute intervals via the jugular catheter. After each administration of bronchoconstrictor the peak resistance value is recorded. Guinea pigs are euthanised with approximately 1.0 mL pentobarbitone sodium (Euthatal) intravenously after the completion of the lung function measurements. Percentage bronchoprotection produced by the compound is calculated at each dose of brochoconstrictor as follows:

$$\% bronchoprotection = \frac{\% {changeR}_{veh} - \% {changeR}_{cmpd}}{\% change {R}_{veh}}$$

Where % change Rᵥₑₕ is the mean of the maximum percentage change in airway resistance in the vehicle treated group.

1.-15. (canceled)

16. A pharmaceutical product comprising, in combination, a first active ingredient which is a muscarinic antagonist selected from:
(R)-3-(1-Phenyl-cycloheptanecarbonyloxy)-1-(pyrazin-2-ylcarbamoylmethyl)-1-azonia-bicyclo[2.2.2]octane X;
(R)-3-(1-Phenyl-cycloheptanecarbonyloxy)-1-(pyridazin-3-ylcarbamoylmethyl)-1-azonia-bicyclo[2.2.2]octane X;
(R)-3-[1-(3-Fluoro-phenyl)-cycloheptanecarbonyloxy]-1-(pyrazin-2-ylcarbamoylmethyl)-1-azonia-bicyclo[2.2.2]octane X;
(R)-3-[1-(3-Fluoro-phenyl)-cycloheptanecarbonyloxy]-1-(isoxazol-3-ylcarbamoylmethyl)-1-azonia-bicyclo[2.2.2]octane X;
(R)-3-(1-Phenyl-cycloheptanecarbonyloxy)-1-(pyridin-2-ylcarbamoylmethyl)-1-azonia-bicyclo[2.2.2]octane X;
(R)-1-[(5-Fluoro-pyridin-2-ylcarbamoyl)-methyl]-3-(1-phenyl-cycloheptanecarbonyloxy)-1-azonia-bicyclo [2.2.2]octane X;
(R)-3-(1-Phenyl-cycloheptanecarbonyloxy)-1-(pyridin-3-ylcarbamoylmethyl)-1-azonia-bicyclo[2.2.2]octane X; and
(R)-1-[(2-Methy 1-pyridin-4-ylcarbamoyl)-methy 1]-3-(1-phenyl-cycloheptanecarbonyloxy)-1-azonia-bicyclo[2.2.2]octane X;
wherein X represents a pharmaceutically acceptable anion of a mono or polyvalent acid, and a second active ingredient which is a β₂-adrenoceptor agonist.

17. A product according to claim 16 wherein the first active ingredient is a muscarinic antagonist which is a 2,5-dichlorobenzene sulphonate or 1-hydroxynaphthalene-2-sulphonate.

18. A product according to claim 16 wherein the first active ingredient is a muscarinic antagonist which is a 1-hydroxynaphthalene-2-sulphonate salt.

19. A product according to claim 16 wherein the first active ingredient is a muscarinic antagonist which is a bromide salt.

20. A product according to claim 16, wherein the β₂-adrenoceptor agonist is formoterol.

21. A product according to claim 16, wherein the β₂-adrenoceptor agonist is selected from:
N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxyl]propanamide,
N-[2-(Diethylamino)ethyl]-N-(2-{[2-(4-hydroxy-2-oxo-2,3-dihydro-1,3-benzothiazol-7-yl)ethyl]amino}ethyl)-3-[2-(3-chlorophenyl)ethoxy]propanamide, and
7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one,
or a pharmaceutically acceptable salt thereof.

22. A product according to claim 16, wherein the β₂-adrenoceptor agonist is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof.

23. A product according to claim 16, comprising, in combination, a first active ingredient which is (R)-3-(1-Phenyl-cycloheptanecarbonyloxy)-1-(pyridin-2-ylcarbamoylmethyl)-1-azonia-bicyclo[2.2.2]octane X wherein X represents a pharmaceutically acceptable anion of a mono or polyvalent acid, and a second active ingredient which is N-[2-(Diethylamino)ethyl]-N-(2-{[2-(4-hydroxy-2-oxo-2,3-dihydro-1,3-benzothiazol-7-yl)ethyl]amino}ethyl)-3-[2-(1-naphthyl)ethoxy]propanamide or a pharmaceutically acceptable salt thereof.

24. A product according to claim 16, comprising, in combination, a first active ingredient which is (R)-3-(1-Phenyl-cycloheptanecarbonyloxy)-1-(pyridin-2-ylcarbamoylmethyl)-1-azonia-bicyclo[2.2.2]octane X wherein X represents a pharmaceutically acceptable anion of a mono or polyvalent acid, and a second active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof.

25. A product according to claim 16, comprising, in combination, a first active ingredient which is (R)-3-(1-Phenyl-cycloheptanecarbonyloxy)-1-(pyridin-2-ylcarbamoylmethyl)-1-azonia-bicyclo[2.2.2]octane X wherein X represents a pharmaceutically acceptable anion of a mono or polyvalent acid, and a second active ingredient which is indacaterol.

26. A method of treating a respiratory disease, which method comprises simultaneously, sequentially or separately administering:
(a) a therapeutically effective dose of a first active ingredient which is a muscarinic receptor antagonist as defined in claim 16; and
(b) a therapeutically effective dose of a second active ingredient which is a β₂-adrenoceptor agonist;
to a patient in need thereof

27. A method according to claim 26, wherein said respiratory disease is chronic obstructive pulmonary disease

28. A kit comprising a preparation of a first active ingredient which is a muscarinic receptor antagonist as defined in claim 16, and a preparation of a second active ingredient which is β₂-adrenoceptor agonist and optionally instructions for the simultaneous, sequential or separate administration of the preparations to a patient in need thereof.

29. A pharmaceutical composition comprising, in admixture, a first active ingredient is a muscarinic receptor antagonist as defined in claim 16 and a second active ingredient which is β₂-adrenoceptor agonist.
